(19) United States
(12) Patent Application Publication
(10) Pub. No.: US 2025/0282821 A1
(43) Pub. Date: Sep. 11, 2025

(54) LIPOPOLYSACCHARIDE-BINDING PEPTOIDS, AND COMPOSITIONS AND METHODS OF USE THEREOF

(71) Applicant: Maxwell Biosciences, Inc., Austin, TX (US)

(72) Inventor: Joshua McClure, Austin, TX (US)

(21) Appl. No.: 19/073,381

(22) Filed: Mar. 7, 2025

Related U.S. Application Data
(60) Provisional application No. 63/562,896, filed on Mar. 8, 2024.

(51) Int. Cl. C07K 7/06; A61K 38/00

(52) U.S. Cl. CPC C07K 7/06; A61K 38/00; C07K 2318/20

(57) ABSTRACT

Lipopolysaccharide-binding peptoids, and compositions and methods of use thereof, are described.

### RELATED APPLICATIONS

This application claims the benefit of U.S. Provisional Application No. 63/562,896 filed Mar. 8, 2024, the contents of which is incorporated by reference herein in its entirety.

### TECHNICAL FIELD

The present disclosure relates to the field of biomedical research and pharmaceuticals. In particular, the present disclosure relates to lipopolysaccharide-binding (LPS-binding) peptoid compounds, and compositions and methods of using the LPS-binding peptoid compounds for treating or preventing pathological conditions that are caused by or associated with LPS in humans and animals.

### BACKGROUND

Lipopolysaccharides (LPS) are major components of the outer membrane of Gram-negative bacteria, implicated in various pathological conditions, including but not limited to sepsis, various autoimmune conditions, and inflammation associated with bacterial infections, among others. The development of therapeutic agents capable of neutralizing LPS may improve outcomes in these conditions. However, the development of new and improved therapeutic agents capable of neutralizing LPS is challenging.

### SUMMARY

The present disclosure relates in several embodiments to LPS-binding peptoid compounds, and compositions and methods of use thereof for treating or preventing pathological conditions that are caused by or associated with LPS in humans and animals.

According to a first aspect, the present disclosure relates in several embodiments to a method of treating or preventing a pathological condition in a subject, wherein the pathological condition is caused by or associated with the presence of a lipopolysaccharide (LPS) in the subject. The method comprises administering to the subject an effective amount of one or more peptoid compounds adapted to bind to the LPS.

The method may include the following details, which can be combined with one another in any combinations unless clearly mutually exclusive:

(i) The LPS may be produced by a Gram-negative bacteria selected from the group consisting of Escherichia (e.g., Escherichia coli), Salmonella (e.g., Salmonella enterica), Klebsiella (e.g., Klebsiella pneumoniae), Pseudomonas (e.g., Pseudomonas aeruginosa), Vibrio (e.g., Vibrio cholerae), Helicobacter (e.g., Helicobacter pylori), Neisseria (e.g., Neisseria meningitidis or Neisseria gonorrhoeae), Bordetella (e.g., Bordetella pertussis, Yersinia (e.g., Yersinia pestis, Yersinia enterocolitica, or Yersinia pseudotuberculosis), Haemophilus (e.g., Haemophilus influenzae), Legionella (e.g., Legionella pneumophila), Campylobacter (e.g., Campylobacter jejuni or Campylobacter coli), Acinetobacter (e.g., Acinetobacter baumannii), Brucella (e.g., Brucella abortus, Brucella melitensis, or Brucella suis), Francisella (e.g., Francisella tularensis), Bacteroides, Shigella (e.g., Shigella flexneri, or Shigella sonnei), Burkholderia (e.g., Burkholderia cepacia or Burkholderia pseudomallei), Pasteurella (e.g., Pasteurella multocida), Proteus (e.g., Proteus mirabilis or Proteus vulgaris), Serratia (e.g., Serratia marcescens), Treponema (e.g., Treponema pallidum), Enterobacter (e.g., Enterobacter cloacae or Enterobacter aerogenes), Moraxella (e.g., Moraxella catarrhalis), Fusobacterium (e.g., Fusobacterium nucleatum), Actinobacillus (e.g., Actinobacillus actinomycetemcomitans), Tannerella (e.g., Tannerella forsythia), Prevotella, Fusobacterium (e.g., Fusobacterium nucleatum), Desulfovibrio, and Capnocytophaga.

(ii) The pathological condition may be selected from the group consisting of Rheumatoid Arthritis, Systemic Lupus Erythematosus, Inflammatory Bowel Disease, Type 1 Diabetes, Multiple Sclerosis, Psoriasis, Ankylosing Spondylitis, Sepsis, Septic shock, Acute respiratory distress syndrome, Multiple organ dysfunction syndrome, Endotoxemia, Neuroinflammation (e.g., in Alzheimer's disease, Parkinson's disease, multiple sclerosis, or depression), Periodontal disease, Chronic inflammatory diseases (rheumatoid arthritis, lupus erythematosus, or IBD), Cardiovascular diseases, Liver diseases (e.g., alcoholic liver disease, non-alcoholic fatty liver disease (NAFLD), or liver cirrhosis), Metabolic disorders (e.g., obesity, insulin resistance, and type 2 diabetes), Acute lung injury, Acute kidney injury, Neonatal disorders (e.g., neonatal sepsis or necrotizing enterocolitis), Autoimmune diseases (e.g, systemic lupus erythematosus (SLE), multiple sclerosis (MS), or autoimmune thyroiditis), Gastrointestinal disorders (e.g., irritable bowel syndrome (IBS), inflammatory bowel disease (IBD), or celiac disease), Reproductive disorders (e.g., preterm labor, fetal growth restriction, or infertility), Psychiatric disorders (e.g., depression, anxiety, or schizophrenia), Chronic obstructive pulmonary disease, Chronic kidney disease, end-stage renal disease, Cholestasis, Pulmonary hypertension, Acute pancreatitis, Osteoarthritis, Cardiomyopathy, Alcoholic liver disease, Gastroesophageal reflux disease, Gastrointestinal cancers, Autoimmune hepatitis, Preterm birth, neonatal complications, Asthma, Necrotizing enterocolitis, Non-alcoholic fatty liver disease, non-alcoholic steatohepatitis, liver cirrhosis, hepatocellular carcinoma, Chronic fatigue syndrome, Vascular dementia, Gut-brain axis disorders, Autism spectrum disorder, Obstructive sleep apnea, Interstitial cystitis/bladder pain syndrome, Chronic rhinosinusitis, Amyotrophic lateral sclerosis, Interstitial Lung Disease, Celiac disease, Autoimmune Encephalitis, Gastric Ulcers, Idiopathic Pulmonary Fibrosis, Preeclampsia, Cardiovascular Diseases, Polycystic Ovary Syndrome, Endometriosis, Periodontal Disease, and inflammation associated with bacterial infections.

(iii) The peptoid compound may prevent or decrease activity of LPS in the subject.

(iv) The peptoid compound may prevent, increase, or decrease binding of the LPS to one or more LPS-binding proteins in the subject.

(v) The LPS-binding protein may be selected from the group consisting of LPS-binding protein, CD14, MD-2, Toll-like receptor 4, Soluble CD14, Lipopolysaccharide and beta-1,3-glucan binding protein, Pentraxins, Surfactant proteins, Lipopolysaccharide-binding protein 2, Bactericidal/permeability-increasing protein, Limulus anti-LPS factor, Plasma lipopolysaccharide-binding protein, Ficolins, Mannose-binding lectin, Lymphocyte antigen 96, Cationic Antimicrobial Peptides, Periplasmic Binding Proteins, Scavenger Receptors, Lipid-Binding Proteins, and Tumor Necrosis Factor Receptor Superfamily Member 6.

(vi) The activity of LPS may be decreased by up to 10%, 20%, 30%, 40%, 50%, 60%, 70%, 80%, 90%, 95%, 99%, or more.

(vii) The one or more peptoid compounds may be formulated in a composition comprising the one or more peptoid compounds and one or more pharmaceutically acceptable excipients.

(viii) The composition may be formulated for topical administration, transdermal administration, transmucosal administration, intraperitoneal administration, subcutaneous administration, intramuscular administration, or intravenous administration to the subject.

(ix) The effective amount may be from 1-1000 mg/day, 25-750 mg/day, 50-500 mg/day, or 100-400 mg/day.

(x) The administration may be one, two, three, or four times per day, once per week, once every two weeks, or once per month.

According to a second aspect, the present disclosure relates in several embodiments to a composition for use in a method of treating or preventing a pathological condition in a subject, wherein the pathological condition is caused by or associated with the presence of a lipopolysaccharide (LPS) in the subject. The composition comprises an effective amount of one or more peptoid compounds adapted to bind to the LPS.

### BRIEF DESCRIPTION OF THE DRAWINGS

The present disclosure may be further understood through reference to the attached figures in combination with the detailed description that follows.

FIG. 1 is the molecular structure of peptoid compound MXB-24,656.

FIG. 2 is the molecular structure of peptoid compound MXB-22,510.

FIG. 3 is the molecular structure of peptoid compound MXB-27,369.

FIG. 4 is the molecular structure of peptoid compound MXB-25,605.

FIG. 5 is the molecular structure of peptoid compound MXB-24,816.

FIG. 6 is the molecular structure of peptoid compound MXB-25,739.

### DETAILED DESCRIPTION

The present disclosure relates in several embodiments to peptoid compounds that can bind to LPS and neutralize one or more harmful effects of LPS in a subject, such as in humans and/or animals. Accordingly, in some embodiments according to the present disclosure, LPS-binding peptoids can be used in methods of treating or preventing pathological conditions that are caused by or associated with LPS in a subject. Without intending to be limited by theory, in some embodiments, the peptoid compounds can possess cationic and amphipathic properties, which may confer LPS-binding activity.

In some embodiments, peptoid compounds can mimic the structural aspects of LPS-binding proteins. In some embodiments, peptoid compounds have cationic characteristics, amphipathic characteristics, or cationic and amphipathic characteristics, and an optimal length for interaction with LPS. Accordingly, in some embodiments, peptoid compounds can bind to and neutralize LPS, preventing or decreasing the interaction of LPS with immune cells and the subsequent release of pro-inflammatory cytokines.

The LPS-binding peptoid compounds of the present disclosure can be used to combat the detrimental effects of LPS and can be used in several embodiments as therapeutic and/or prophylactic agents for a range of pathological conditions associated with LPS.

As described in further detail herein, in some embodiments, for example and not by way of limitation, the present disclosure relates to methods of treating or preventing sepsis, autoimmune conditions, and inflammation associated with bacterial infections, among others.

In some embodiments, the present disclosure relates to a method of treating or preventing a pathological condition in a subject, wherein the pathological condition is caused by or associated with the presence of a lipopolysaccharide (LPS) in the subject, comprising: administering to the subject an effective amount of one or more peptoid compounds adapted to bind to the LPS.

LPS are significant outer membrane components of gram-negative bacteria. LPS are large amphipathic glycoconjugates that typically consist of a lipid domain (hydrophobic) attached to a core oligosaccharide and a distal polysaccharide. These molecules are also known as lipogylcans due to the presence of lipid and sugar molecules. The lipopolysaccharides are composed of (1) Lipid A: the hydrophobic domain, which is an endotoxin and the main virulence factor, (2) 0-antigen, the repeating hydrophilic distal oligosaccharide, and (3) the hydrophilic core polysaccharide.

The lipid A component varies from one organism to another and imparts specific pathogenic attributes to the bacteria. Inherent to gram-negative bacteria, LPS provides integrity to the bacterial cell and a mechanism of interaction of the bacteria to other surfaces. Most bacterial LPS molecules are thermostable and generate a robust pro-inflammatory stimulus for the immune system in mammals. Since different types of LPS are present in different genera of gram-negative bacteria, LPS is used for serotyping gram-negative bacteria. More specifically, the O-antigen imparts serological distinction to the bacterial species. Also, the size and composition of LPS are highly dynamic among bacterial species.

The Gram-negative bacterial cell membrane is composed of an outer membrane and an inner membrane. The outer membrane is exposed to the outside environment, while the inner membrane envelops the cytoplasm. The Gram-negative bacterial membrane does not have a phospholipid bilayer but instead has an asymmetric bilayer with LPS on the outside and phospholipid on the inside. In non-capsulated bacterial strains, such as E. coli, Pseudomonas, etc., LPS is exposed to the cell surface, while in the capsulated strains such as Klebsiella pneumonia, Haemophilus influenza, etc., LPS is present below the capsular layer.

Of the three components, lipid A is the most bioactive component of LPS and a potent part of the endotoxin response generated by a molecule. Thus, LPS can be used for the early detection of infection since it induces an innate immune response, specifically through Toll-like receptors (TLRs). Even when the immune system causes lysis of the bacterial cells, lipid A containing fragments of the membrane released in the circulation cause fever, diarrhea, and in adverse circumstances, lead to septic (endotoxic) shock. Though lipid A moiety is a very conserved part of the LPS, the structure differs among different strains, species, and subspecies of bacteria. Hence, the overall immune activation and response depend upon the structure of lipid A moiety of LPS. LPS recognition by the host is crucial for clearing the infections of invading bacterial pathogens. On the other hand, most gram-negative bacteria show innate resistance to many antimicrobial therapies due to the presence of LPS because it develops a permeability barrier at the cell surface.

In contrast to lipid A, O-antigens are the most variable part of the LPS molecule that imparts antigenic specificity to the molecule. The size or the composition of the O-antigen can reliably indicate the virulence potential of a bacterial strain. Modifications in the O-antigen plausibly play an essential role in the infection process. It mainly imparts the potential to induce attachment, colonization of the host, and the ability to circumvent host defense mechanisms.

Canonical Lipid A, such as from non-invading E. coli, are speculated to be agonistic, while less conical lipid A, such as P. gingivalis, may activate a different signal (such as TLR4 instead of TLR2 and the strictly cylindrical lipid A, as Rhodobacter sphaeroides, tend to be antagonistic to TLRs. Hence, more pathogenic bacteria have evolved their lipid A moiety to evade host immune attacks.

LPS are present in a wide range of Gram-negative bacterial species belonging to the phyla Proteobacteria, Bacteroidetes, and Spirochaetes, among others. Some example bacterial genera known to have lipopolysaccharides include the following, without limitation:

Escherichia: Including Escherichia coli, a well-studied model organism and common inhabitant of the human gastrointestinal tract.

Salmonella: Including pathogenic species such as Salmonella enterica, which can cause foodborne illness in humans.

Klebsiella: Including Klebsiella pneumoniae, which can cause pneumonia, urinary tract infections, and other infections in humans.

Pseudomonas: Including Pseudomonas aeruginosa, an opportunistic pathogen associated with hospital-acquired infections.

Vibrio: Including Vibrio cholerae, the causative agent of cholera, a severe diarrheal disease.

Helicobacter: Including Helicobacter pylori, which colonizes the human stomach and is associated with gastritis, peptic ulcers, and gastric cancer.

Neisseria: Including Neisseria meningitidis and Neisseria gonorrhoeae, which can cause meningitis and gonorrhea, respectively.

Bordetella: Including Bordetella pertussis, the causative agent of whooping cough (pertussis).

Yersinia: Including Yersinia pestis, the bacterium responsible for causing plague, as well as Yersinia enterocolitica and Yersinia pseudotuberculosis, which can cause gastrointestinal infections in humans.

Haemophilus: Including Haemophilus influenzae, a common cause of respiratory tract infections, including pneumonia and otitis media.

Legionella: Including Legionella pneumophila, the bacterium responsible for causing Legionnaires' disease, a severe form of pneumonia.

Campylobacter: Including Campylobacter jejuni and Campylobacter coli, which are major causes of bacterial gastroenteritis in humans.

Acinetobacter: Including Acinetobacter baumannii, an opportunistic pathogen associated with healthcare-associated infections, particularly in immunocompromised individuals.

Brucella: Including Brucella abortus, Brucella melitensis, and Brucella suis, which can cause brucellosis, a zoonotic disease transmitted from animals to humans.

Francisella: Including Francisella tularensis, the bacterium responsible for causing tularemia, a potentially severe infectious disease.

Bacteroides: Bacteroides species are common inhabitants of the human gut microbiota and are known to produce LPS.

Shigella: Shigella species, including Shigella flexneri, Shigella sonnei, and others, are responsible for causing shigellosis, a diarrheal disease.

Burkholderia: Burkholderia species, such as Burkholderia cepacia and Burkholderia pseudomallei, are opportunistic pathogens associated with respiratory infections and melioidosis, respectively.

Pasteurella: Pasteurella species, including Pasteurella multocida, are often found as normal flora in the respiratory tracts of animals and can cause infections in humans, particularly following animal bites or scratches.

Proteus: Proteus species, such as Proteus mirabilis and Proteus vulgaris, are common causes of urinary tract infections and other nosocomial infections.

Serratia: Serratia species, including Serratia marcescens, are opportunistic pathogens that can cause various infections, including respiratory and urinary tract infections.

Treponema: Treponema species, such as Treponema pallidum, are responsible for causing syphilis and other treponemal diseases.

Enterobacter: Enterobacter species, including Enterobacter cloacae and Enterobacter aerogenes, are opportunistic pathogens associated with healthcare-associated infections.

Moraxella: Moraxella species, including Moraxella catarrhalis, can cause respiratory tract infections, particularly in individuals with underlying respiratory conditions.

Fusobacterium: Fusobacterium species, such as Fusobacterium nucleatum, are anaerobic bacteria commonly found in the oral cavity and gastrointestinal tract, and they are associated with various infections including periodontal disease and certain types of abscesses.

Actinobacillus: Actinobacillus species, such as Actinobacillus actinomycetemcomitans, are associated with periodontal disease in humans and animals.

Tannerella: Tannerella forsythia is a Gram-negative bacterium implicated in periodontal disease and is often found in conjunction with other periodontal pathogens.

Prevotella: Prevotella species are Gram-negative anaerobic bacteria that can be found in various parts of the body including the oral cavity, gastrointestinal tract, and urogenital tract. Some species are associated with periodontal disease and other infections.

Fusobacterium: Fusobacterium species, such as Fusobacterium nucleatum, are anaerobic bacteria commonly found in the oral cavity and gastrointestinal tract, and they are associated with various infections including periodontal disease and certain types of abscesses.

Desulfovibrio: Desulfovibrio species are sulfate-reducing bacteria found in various environments including the human gut, and they are associated with inflammatory bowel disease and other conditions.

Capnocytophaga: Capnocytophaga species are found in the oral cavity and can cause infections such as periodontitis, cellulitis, and bacteremia.

The above are non-limiting examples of Gram-negative bacterial genera that contain LPS in their outer membrane. Other bacterial that also produce LPS are identifiable by skilled persons upon reading the present disclosure.

Accordingly, in some embodiments, the LPS may be from any Gram-negative bacterium, such as those described herein, without limitation, among others.

LPS is a serologically reactive bacterial toxin, and as little as 1 to 2 mg entered intravenously can be lethal. LPS can enter the bloodstream through intestinal absorption of the LPS produced by gut bacteria. LPS can induce toxicity if it reaches the basal side of the gut epithelium, which has exposure to the deeper tissues. The body has developed compartmentalization to prevent high amounts of LPS from entering the bloodstream. However, gut lesions or a diet rich in lipids facilitate transport across the membrane into the systemic circulation. LPS is a potent pyrogen for which the immune system mounts an immediate response. Many food products, supplements, and probiotics can pose a health risk as they contain gram-negative bacteria or LPS. Though these products are subject to digestion upon oral intake, it represents a health risk for patients with gastrointestinal disease. Furthermore, LPS can enter the human system through pharmaceutical preparations such as parenteral drug products, which can activate the complement system by the alternative pathway and can result in death.

A weak immune system is another breach in the barrier that leads to the endotoxic effects of LPS during an infection process. If allowed to progress unabated, it can lead to serious consequences such as septic shock or hypotension. Bacterial modifications in LPS structure, mostly the Lipid A portion, are a sophisticated strategy employed by Gram-negative bacteria to adapt to the host environment. Certain types of autoimmune diseases and allergies may be associated with LPS. A large cohort study conducted on the fecal samples from North European infants indicates that the presence of Bacteroides species LPS is associated with higher levels of food allergy and anti-insulin antibodies, indicating early signs of immune dysfunction.

Besides lipid-binding protein (LBP), lipopolysaccharide-binding proteins are carriers for LPS in the blood. They can transfer LPS either to the macrophages or to the serum carrier lipoproteins (HDL and LDL). The transfer of LPS to macrophages initiates signal transduction to induce proinflammatory cytokines, while the delivery of LPS to HDL or LDL compromises the immune reaction against the infection. Additionally, the binding of LPS to lipoproteins induces dyslipidemia

LPS, the major glycolipid of the outer membrane in gram-negative bacteria, is stabilized by divalent cations that increase the overall negative charge to the membrane. Lipid A anchors the molecule to the outer membrane, the core oligosaccharide that is integral to imparting and maintaining membrane integrity, and the O-antigen polysaccharide that is connected to the core oligosaccharide as is in direct contact with the external environment.

Overall, LPS structurally provides an effective permeability barrier against molecules that could be harmful to the bacterial cell. The LPS molecules only including lipid A and core oligosaccharides, are generally referred to as “rough” and often called lipooligosaccharides, while the complete LPS capped with O antigen is referred to as “smooth”. The only gram-positive bacteria to contain an authentic lipopolysaccharide is Listeria monocytogenes.

Most of the commensal and pathogenic gram-negative bacteria have been shown to form biofilms. These biofilms provide stability to the bacterial cell and resilience to these bacterial populations against various drugs and antibiotics. LPS modification through palmitoylation is one of the strategies that lead to stable biofilm formation. Bacteria such as E. coli and Pseudomonas aeruginosa display increased incorporation of palmitate acyl chain in the lipid A moiety. Palmitic acid imparts increased hydrophobicity to LPS, which is inherent to biofilms forming over both biotic and abiotic surfaces. Biofilms are refractory to drug therapy, and thus bacteria can develop a resistant phenotype in vivo. Biofilms also accumulate various nutrients, such as amino acids and antiadhesion molecules. Biofilms also demonstrate increased tolerance to host immune responses, which enhance bacterial survival in vivo. Many hospital-acquired or resistant infections, such as catheter infections predominantly caused by Escherichia coli, Pseudomonas aeruginosa, Staphylococcus aureus, etc., show higher resistance to treatment due to their ability to form biofilms. Besides precluding host defense, the biofilm matrix also provides antimicrobial resistance by subduing antibiotic penetration and altering the host microenvironment.

LPS biogenesis employs the process of assembly at the bacterial inner membrane and subsequent translocation to the bacterial cell surface. The hydrophobic Lipid A part of the molecule is an acylated β-1′-6-linked glucosamine disaccharide, which forms the outer leaflet of the outer membrane. These glucosamines are acylated at the 2, 3, 2′ and 3′ positions and phosphorylated at the 1 and 4′ positions, which typically gives a hexa-acylated feature to the mature Lipid A. Hexacylated, bisphosphorylated lipid A as seen in E. coli and Salmonella is more immunogenic than other forms of lipid A. Secondly, the core oligosaccharide is a non-repeating polymer linked to lipid A through the glucosamine moiety. It is inherently composed of 3-deoxy-D-manno-oct-2-ulosonic acid (Kdo) residues, various hexoses, and heptoses. In some bacterial strains, the sugars become substituted with phosphates, phosphoethanolamine, etc. Thirdly, 0-antigen is the most variable part of LPS imparting immunogenic properties to the LPS. It lies towards the inner leaflet of the outer membrane and is attached to the core polysaccharide. It is a repeating oligosaccharides unit of two to eight sugars. There are few bacterial strains, such as Neisseria, that do not synthesize O-antigen. However, the presence of O-antigens is necessary for the adherence specificity of the bacteria to tissues.

The overall structure of the LPS is conserved. However, considerable structural differences lead to variations at the species and strain level.

The primary function of LPS is to provide structural integrity and a permeability barrier to protect the bacterial cell from the entry of deleterious molecules such as toxins and bile salts during its inhabitation in the gastrointestinal tract. The presence of a large number of saturated fatty acid moieties leads to extensive interaction within the acyl chain, which results in low fluidity of the membrane bilayer. The high negativity of the membrane due to the presence of the phosphate group becomes stabilized by divalent cations such as Mg, which intercalate between LPS molecules. The final structure formed by polyionic interactions enhances LPS packing, finally making LPS a structural barrier to the bacterial cell.

LPS is the primary component that imparts pathogenicity to the bacterial cell. Compared to the classical bacterial exotoxins, the LPS are more stable and are the primary biological response modifiers to induce specific symptoms and pathologies of the diseases. LPS exerts a potent and pleiotropic stimulus of host immune cells. The immune system recognizes the Lipid A component of LPS, which is released from the dividing cells in soluble form, from the lysed bacterial cells upon autolysis, or killing by complement activation or phagocytosis or by the effect of antibiotics. The primary response is through the identification and binding of the lipid A component to the TLR4 of the host cell. Since Lipid A is can be variable in its composition, different strains of bacteria mount different levels of the immune response. Lipid A produced by E. coli and Salmonella is highly immunogenic, while others, such as that of Yersinia pestis, modulate the extent of acylation of lipid A upon infection to produce LPS of low immunogenicity in vivo.

The O-antigen of the LPS imparts antigenicity to the bacterial cell leading to the production of antibodies. Nonetheless, the variability in the length of the O-antigen chain can prevent complement-mediated bacterial controls and killing. O antigen also contributes to pathogen evasion of phagocytosis by immune cells. Another essential function of LPS is in biofilm formation. The clustering of bacteria into a biofilm is induced by LPS that helps colonization and development of a drug-resistant phenotype that is refractory to most antibiotic therapies.

Inside the host system, the lipid A moiety of LPS is detected at picomolar levels by the receptor on the surface of macrophages and endothelial cells. Initially, LPS binds to the LBP in the serum, which transfers it to the CD14 receptor present on the cell membrane of the immune cells. The CD14 transfers it to the MD2 (a non-anchored protein), which interacts with Toll-like receptor-4 (TLR4). Thus, LPS binds with the CD14/TLR4/MD2 receptor complex present in many host cell types, such as monocytes, dendritic cells, macrophages, and B cells. The subsequent response depends on the cell type to which LPS is bound. In monocyte and macrophages, three plausible responses are triggered: (1) Production of cytokines like TNF, IL-1, IL-6, IL-8, which stimulates prostaglandins and leukotrienes release, finally leading to the inflammation and septic shock that are the significant features of endotoxemia. (2) Complement activation initiates histamine release causing vasodilation with neutrophil chemotaxis and accumulation. (3) Activation of coagulation cascade: Blood-clotting Factor XII activates humoral systems leading to coagulation, thrombosis, acute disseminated intravascular coagulation. Plasmin activation leads to fibrinolysis and hemorrhage; activation of the alternative pathway leads to inflammation. Activation of kinin releases bradykinins and other vasoactive peptides, causing hypotension. Finally, this induces the development of inflammation, hemorrhage, intravascular coagulation, and septic shock.

LPS is a virulence factor, and based on its structure and function, is used to classify bacteria in serogroups. Thus, it serves as a pathogen-specific biomarker to aid in serological discrimination of Gram-negative bacteria. Timely identification and characterization of pathotypes may be important for the early mitigation and treatment of infections. Being the primary immune stimulator is in host cells, it serves as an early indicator of acute infection. Thus, LPS testing can be more specific than other serological assays. The present methods of LPS testing are generally sensitive, but many cannot differentiate between LPS serogroups. Also, the amphiphilic property of LPS projects a critical bottleneck to the sensitivity and ease of use of the assays. The usual detection methods depend on lipid A antigen detection. However, LPS detection through lipid-A limits its ability to accurately identify a bacterial species because Lipid A is highly conserved among species and serotypes. The testing methods divide into six overlapping categories: in vivo and in vitro tests, modified immunoassays, biological assays, and chemical assays.

The Limulus Amoebocyte Lysate (LAL) Assay utilizes the property of amoebocytes from Limuluspolyphemus to agglutinate upon the addition of endotoxin through a protease cascade reaction. The detection method uses the lysates of amoebocytes. LAL is the gold standard for the detection of lipid A. Nevertheless, this assay is subject to variability and inhibition through several chemical reactions. Variants of the LAL assay use chromogenic, turbidimetric, or viscometric methods to measure the degree of clotting. The sensitivity of this assay method depends on the sample type, processing method, time, and the dilution factor. The LAL assay is used in urine, cerebral spinal fluid, synovial fluid, ascites fluid, vaginal and cervical fluids, bronchoalveolar lavage samples, and even seawater.

Biological and Chemical-based LPS Sensing technologies use biosensors that are activated with proteins or molecules to pull down LPS from a sample. The natural carriers for LPS, such as LBP or serum carrier proteins such as HDL and LDL, as well as synthetic aptamers, peptides, and metal-cation complex, are used to bind and pull down LPS in these assay methods. Different types of signals serve to detect and enhance the readout of the LPS signal. Electrochemical (EC) sensing requires a recognition ligand and a transducer to measure the variation in signal. Fluorescence-based sensing needs a receptor that captures LPS, and another molecule emits a fluorescent signal upon binding to the antigen. Aptamers attached to gold nanoparticles have been used to detect LPS using EIS. This technique has an enhanced detection limit of 0.1 pg/mL. Aptamers are also being used in a magnetic aptasensor to detect LPS. The use of lipid complexes (liposomes) has also been useful as a testing mechanism. This method manipulated the amphipathic nature of LPS. These biosensors provide high sensitivity, up to picogram or femtomolar range. However, these assays are incapable of discriminating between LPS serogroups.

Immunoassays for LPS Detection and Antibody Selection can be used to detect LPS. Enzyme-linked Immunosorbent assays (ELISA), based on the reaction between antigen and antibody, have been used to test LPS. However, the amphipathic nature of LPS leads to inconsistent binding on ELISA plates and variable conformations of epitope binding sites. This inconsistency resulted in low sensitivity and reproducibility of ELISA for LPS. There are two basic types of LPS-ELISAs, one which detects LPS antigen, and the other detects LPS antibody titers. An enhanced sandwich ELISA form that minimizes the low sensitivity and reproducibility associated with LPS-ELISA was developed and commercialized as ENDOLisa. It has a sensitivity between 0.05 and 500 EU/mL.

The second type of ELISA format is designed to detect antibodies against LPS. The plate surface is coated with the antigen to pull down antibodies such as IgA, IgG, etc., from serum. This method has its basis in testing the adaptive immune responses; hence it cannot be utilized to check the initial exposure to the pathogen. This type of ELISA format is useful to test population health risks and monitor epidemiology. The major drawback of LPS detection is that many LPS antigens have not been isolated yet. Thus screening methods are only present for the strains for which antigens are available. Alternative methods for antibody screening utilized immunoblotting and flow cytometry. Methods of LPS testing with the above techniques are affected by endogenous endotoxins present on glassware, plastics, or reagents, potentially contributing to false-positive results.

In addition to medical diagnostics, LPS detection also provides a method for detecting Escherichia coli in the food industry, an organism often associated with food-borne illnesses. The successful detection methods for LPS couple sensitive detection platforms with surfaces designed to maximize the binding of amphiphilic PAMPs. Monitoring LPS levels in pharmaceutical products and medical devices is typically done using LAL assays.

LPS is a potent endotoxin that binds to cell surface receptors such as TLR4/CD14/MD2 that induces the secretion of proinflammatory cytokines, nitric oxide, and eicosanoids. The presence of LPS in the blood or interstitial fluid can lead to endotoxemia through Lipid A moiety, which can cause septic shock under exaggerated immune response. Septic shock includes tachycardia, tachypnea, temperature modulations, and coagulation cascade activation, leading to arterial and venous dilation. The resulting hypovolemia leads to cellular dysfunction as a result of inadequate tissue perfusion. LPS exposure may correlated with autoimmune diseases, and allergies while high concentrations of LPS in the blood lead to metabolic syndrome. This increases the risk of serious diseases such as type 2 diabetes, heart diseases, and liver diseases. Furthermore, endotoxins are inherently responsible for the clinical manifestations of infections with Gram-negative bacterial pathogens, such as Neisseria meningitides, that cause meningococcal disease, such as Waterhouse-Friderichsen syndrome, meningococcemia, and meningitis. Specific opportunistic pathogens such as Pseudomonas aeruginosa, Burkholderia epacian complex bacteria, Helicobacter pylori, and Salmonella enterica, among others, can adapt through LPS structure-function changes to develop a chronic infection in the respiratory and gastrointestinal tract.

LPS can induce membrane lipid disturbances, which affect cholesterol interacting proteins, lipoprotein metabolism, and membrane apo E/amyloid-beta interactions. These alterations predispose to hypercholesterolemia, dyslipidemia, and non-alcoholic fatty liver disease. In some cases, the presence of LPS can interfere with the clearance of toxins from the body linking it to neurological degeneration.

LPS is a powerful toxin that, when in the body, can trigger inflammation by binding to cell receptors. Excessive LPS in the blood can lead to endotoxemia, potentially causing a harmful condition called septic shock. This condition includes symptoms like rapid heart rate, quick breathing, temperature changes, and blood clotting issues, resulting in blood vessels widening and reduced blood volume, leading to cellular dysfunction.

Even low levels of LPS exposure may be associated with autoimmune diseases and allergies. High levels of LPS in the blood can lead to metabolic syndrome, increasing the risk of conditions like diabetes, heart disease, and liver problems.

LPS also plays a crucial role in symptoms caused by infections from harmful bacteria, including severe conditions such as Waterhouse-Friderichsen syndrome, meningococcemia, and meningitis, among others. Certain bacteria can adapt their LPS to cause long-lasting infections in the respiratory and digestive systems.

LPS disrupts cell membrane lipids, affecting cholesterol and metabolism, potentially leading to high cholesterol, abnormal blood lipid levels, and non-alcoholic fatty liver disease. In some cases, LPS can interfere with toxin clearance, which may be linked to neurological issues.

In general, the health effects of LPS are due to its abilities as a potent activator and modulator of the immune system, especially its inducement of inflammation.

The presence of endotoxins in the blood is called endotoxemia. High level of endotoxemia can lead to septic shock, while lower concentration of endotoxins in the bloodstream is called metabolic endotoxemia. Endotoxemia is associated with obesity, diet, cardiovascular diseases, and diabetes.

Moreover, endotoxemia of intestinal origin, especially, at the host-pathogen interface, is considered to be an important factor in the development of alcoholic hepatitis, which is likely to develop on the basis of the small bowel bacterial overgrowth syndrome and an increased intestinal permeability.

Lipid A may cause uncontrolled activation of mammalian immune systems with production of inflammatory mediators that may lead to septic shock. This inflammatory reaction is mediated by Toll-like receptor 4 which is responsible for immune system cell activation. Damage to the endothelial layer of blood vessels caused by these inflammatory mediators can lead to capillary leak syndrome, dilation of blood vessels and a decrease in cardiac function and can lead to septic shock. Pronounced complement activation can also be observed later in the course as the bacteria multiply in the blood. High bacterial proliferation triggering destructive endothelial damage can also lead to disseminated intravascular coagulation (DIC) with loss of function of certain internal organs such as the kidneys, adrenal glands and lungs due to compromised blood supply. The skin can show the effects of vascular damage often coupled with depletion of coagulation factors in the form of petechiae, purpura and ecchymoses. The limbs can also be affected, sometimes with devastating consequences such as the development of gangrene, requiring subsequent amputation. Loss of function of the adrenal glands can cause adrenal insufficiency and additional hemorrhage into the adrenals causes Waterhouse-Friderichsen syndrome, both of which can be life-threatening.

Neisseria gonorrhoeae LPS can cause damage to human fallopian tubes.

In auto-immune disease, the molecular mimicry of some LPS molecules is thought to cause autoimmune-based host responses, such as flareups of multiple sclerosis. Other examples of bacterial mimicry of host structures via LPS are found with the bacteria Helicobacter pylori and Campylobacter jejuni, organisms which cause gastrointestinal disease in humans, and Haemophilus ducreyi which causes chancroid. Certain C. jejuni LPS serotypes (attributed to certain tetra- and pentasaccharide moieties of the core oligosaccharide) have also been implicated with Guillain-Barré syndrome and a variant of Guillain-Barré called Miller-Fisher syndrome.

Increased endotoxin load, which can be a result of increased populations of endotoxin-producing bacteria in the intestinal tract, is associated with certain obesity-related patient groups. Purified endotoxin from Escherichia coli can induce obesity and insulin-resistance when injected into germ-free mouse models. Enterobacter cloacae LPS may contribute to obesity and insulin resistance LPS may induce an inflammation-mediated pathway leading to obesity and insulin resistance. Bacterial genera associated with endotoxin-related obesity effects include Escherichia and Enterobacter.

LPS might play a role in depression. Administration of LPS in mice can lead to depressive symptoms, and there may be elevated levels of LPS in people with depression. Inflammation may sometimes play a role in the development of depression, and LPS is pro-inflammatory.

Inflammation induced by LPS can induce cellular senescence, as has been shown for the lung epithelial cells and microglial cells (the latter leading to neurodegeneration).

In addition to the non-limiting examples discussed above, numerous pathological conditions are associated with LPS, such as the non-limiting examples discussed below.

LPS can contribute to the initiation and exacerbation of various autoimmune inflammatory disorders, particularly through their role in triggering systemic inflammation and immune dysregulation. The direct causation of these diseases by LPS is complex and may involve a combination of genetic, environmental, and immunological factors. However, LPS is recognized for its potential to exacerbate inflammation in several autoimmune conditions, such as the following:

Rheumatoid Arthritis (RA): LPS may amplify inflammation in joints, contributing to the disease's progression. LPS exposure can activate the innate immune system, leading to the production of pro-inflammatory cytokines and the recruitment of immune cells to the synovium. This inflammatory response can contribute to the development of synovitis, cartilage degradation, and bone erosion in affected joints. Additionally, LPS-induced inflammation may perpetuate the autoimmune response in RA by promoting the production of autoantibodies and the activation of autoreactive T cells. Therefore, LPS may play a role in the initiation and progression of rheumatoid arthritis.

Systemic Lupus Erythematosus (SLE): LPS can exacerbate systemic and organ-specific inflammation, affecting disease severity. LPS exposure can activate the immune system and trigger the production of autoantibodies against self-antigens, leading to immune complex deposition and tissue damage. Additionally, LPS-induced inflammation can exacerbate disease activity in individuals with SLE, contributing to the development of systemic manifestations such as arthritis, nephritis, skin rashes, and vasculitis. Moreover, increased intestinal permeability and gut dysbiosis, which may result in higher LPS levels in the circulation, have been observed in patients with SLE, suggesting a potential role of LPS in disease pathogenesis.

Inflammatory Bowel Disease (IBD), including Crohn's Disease and Ulcerative Colitis: LPS can worsen intestinal inflammation and barrier dysfunction. LPS has been implicated in the pathogenesis of inflammatory bowel disease, including Crohn's disease and ulcerative colitis. LPS, derived from the gut microbiota, can activate immune cells in the intestinal mucosa, leading to chronic inflammation and tissue damage. In genetically susceptible individuals, dysregulated immune responses to LPS may contribute to the development and perpetuation of IBD. Additionally, alterations in the gut microbiota composition and increased intestinal permeability, which can result in higher levels of circulating LPS, have been observed in patients with IBD, suggesting a potential role of LPS-mediated gut dysbiosis and immune activation in the pathogenesis of this condition.

Type 1 Diabetes (T1D): LPS exposure has been implicated in the inflammation of pancreatic islets, potentially accelerating beta-cell destruction. Evidence suggests that LPS-induced inflammation, intestinal permeability, gut microbiota dysbiosis, and genetic susceptibility may all play a role in the development of T1D.

Multiple Sclerosis (MS): LPS can stimulate the immune system in a way that may worsen neuroinflammation and demyelination. Evidence suggests that LPS-induced inflammation, BBB dysfunction, gut microbiota dysbiosis, and genetic susceptibility may all play a role in the development or exacerbation of MS.

Psoriasis: LPS can contribute to the systemic inflammatory state, potentially triggering or worsening skin lesions. There is evidence suggesting a potential link between LPS and psoriasis, a chronic inflammatory skin disorder characterized by red, scaly patches and plaques. LPS from the gut microbiota or bacterial colonization on the skin may trigger an immune response in genetically predisposed individuals, leading to the development or exacerbation of psoriasis. LPS-induced activation of the innate immune system can promote the production of pro-inflammatory cytokines and chemokines, recruitment of immune cells to the skin, and proliferation of keratinocytes, contributing to the inflammatory processes and epidermal hyperplasia characteristic of psoriatic lesions. Additionally, increased levels of circulating LPS and alterations in the gut microbiota composition have been reported in patients with psoriasis, suggesting a potential role of LPS-mediated gut inflammation and dysbiosis in the pathogenesis of this condition.

Ankylosing Spondylitis: LPS may promote inflammation in the vertebral joints, leading to increased symptoms. There is evidence suggesting that LPS may play a role in the pathogenesis of ankylosing spondylitis, a chronic inflammatory disease primarily affecting the spine and sacroiliac joints. LPS-induced inflammation in the gut mucosa and subsequent translocation of LPS into the bloodstream may trigger an immune response in genetically predisposed individuals, leading to the development of AS. LPS-mediated activation of the innate immune system can promote the production of pro-inflammatory cytokines and the recruitment of immune cells to the joints, contributing to synovitis, enthesitis, and bone remodeling characteristic of AS. Additionally, increased levels of circulating LPS and alterations in the gut microbiota composition have been observed in patients with AS, suggesting a potential role of LPS-mediated gut inflammation in the pathogenesis of this condition.

Some rare autoinflammatory disorders, such as familial Mediterranean fever (FMF) and cryopyrin-associated periodic syndromes (CAPS), involve dysregulated innate immune responses characterized by excessive production of pro-inflammatory cytokines. While the exact role of LPS in these conditions is not fully understood, aberrant activation of the innate immune system by endotoxin or other microbial products may contribute to the pathogenesis of autoinflammatory disorders.

These associations between LPS and autoimmune diseases highlight the role of microbial components in modulating immune responses and exacerbating inflammatory processes.

Pathological conditions associated with LPS also include the following, without limitation:

Sepsis: LPS is a major contributor to the development of sepsis, a life-threatening condition characterized by systemic inflammation resulting from the body's response to infection. Excessive LPS triggers an overwhelming immune response, leading to widespread inflammation, organ dysfunction, and potentially death if not promptly treated.

Septic shock: In severe cases of sepsis, the immune response triggered by LPS can lead to septic shock. Septic shock is characterized by a profound drop in blood pressure, which can result in inadequate blood flow to vital organs, leading to organ failure and death if not promptly treated.

Acute respiratory distress syndrome (ARDS): LPS-induced sepsis can lead to the development of ARDS, a severe lung condition characterized by inflammation and fluid buildup in the lungs. ARDS can cause severe respiratory failure and is associated with a high mortality rate.

Multiple organ dysfunction syndrome (MODS): Excessive LPS-induced inflammation can lead to dysfunction in multiple organs, resulting in MODS. This condition is characterized by the failure of two or more organ systems, such as the lungs, kidneys, liver, or heart, and is associated with a high mortality rate.

Endotoxemia: Endotoxemia refers to the presence of endotoxins, including LPS, in the bloodstream. It can occur in conditions such as sepsis or following the administration of certain medications or procedures. Endotoxemia can lead to systemic inflammation and organ dysfunction.

Neuroinflammation: LPS-induced systemic inflammation can also affect the central nervous system, leading to neuroinflammation. This neuroinflammatory response has been implicated in the pathogenesis of conditions such as Alzheimer's disease, Parkinson's disease, and multiple sclerosis, and depression.

Periodontal disease: LPS produced by oral bacteria, particularly Gram-negative bacteria found in dental plaque, can contribute to the development and progression of periodontal disease, a chronic inflammatory condition affecting the gums and supporting structures of the teeth.

Chronic inflammatory diseases: LPS has been implicated in the pathogenesis of various chronic inflammatory diseases, including rheumatoid arthritis, lupus erythematosus, and inflammatory bowel disease (IBD). In these conditions, the presence of LPS can contribute to sustained inflammation and tissue damage.

Cardiovascular diseases: LPS-induced inflammation has been linked to the development and progression of cardiovascular diseases such as atherosclerosis, coronary artery disease, and myocardial infarction. Chronic exposure to LPS can promote endothelial dysfunction, vascular inflammation, and plaque formation, increasing the risk of cardiovascular events.

Liver diseases: LPS is cleared from the bloodstream by the liver, but in conditions such as alcoholic liver disease, non-alcoholic fatty liver disease (NAFLD), and liver cirrhosis, impaired liver function can lead to increased systemic levels of LPS. Elevated LPS levels can exacerbate liver inflammation and contribute to the progression of liver disease.

Metabolic disorders: There is growing evidence linking LPS to metabolic disorders such as obesity, insulin resistance, and type 2 diabetes. LPS-induced inflammation can disrupt insulin signaling pathways, promote adipose tissue inflammation, and contribute to the development of metabolic dysfunction.

Acute lung injury (ALI) and acute respiratory distress syndrome (ARDS): In addition to ARDS, LPS can also contribute to the development of ALI, a less severe form of lung injury characterized by inflammation and increased permeability of the alveolar-capillary barrier. ALI can progress to ARDS in severe cases, leading to respiratory failure.

Acute kidney injury (AKI): LPS-induced systemic inflammation can contribute to the development of AKI, a sudden decline in kidney function often seen in critically ill patients, particularly those with sepsis or septic shock. The inflammatory response triggered by LPS can lead to renal vasoconstriction, tubular injury, and impaired kidney function.

Neonatal disorders: LPS exposure during pregnancy or in the neonatal period can lead to neonatal sepsis, a serious infection in newborns associated with high morbidity and mortality. LPS-induced inflammation can also contribute to other neonatal disorders such as necrotizing enterocolitis (NEC), a gastrointestinal disease characterized by inflammation and tissue necrosis in premature infants.

Autoimmune diseases: While the exact role of LPS in autoimmune diseases is not fully understood, there is evidence to suggest that LPS exposure may trigger or exacerbate autoimmune responses in susceptible individuals. Conditions such as systemic lupus erythematosus (SLE), multiple sclerosis (MS), and autoimmune thyroiditis have been associated with dysregulated immune responses involving LPS.

Gastrointestinal disorders: LPS has been implicated in the pathogenesis of various gastrointestinal disorders, including irritable bowel syndrome (IBS), inflammatory bowel disease (IBD), and celiac disease. Increased intestinal permeability, bacterial translocation, and immune activation by LPS may contribute to gastrointestinal inflammation and symptoms.

Reproductive disorders: LPS exposure has been linked to reproductive disorders such as preterm labor, fetal growth restriction, and infertility. In pregnant women, LPS-induced inflammation can disrupt placental function and fetal development, increasing the risk of adverse pregnancy outcomes.

Psychiatric disorders: There is emerging evidence suggesting a potential link between LPS and psychiatric disorders such as depression, anxiety, and schizophrenia. Chronic low-grade inflammation, triggered in part by LPS, has been implicated in the pathophysiology of these conditions. LPS-induced neuroinflammation may disrupt neurotransmitter function and neuronal signaling, contributing to the development or exacerbation of psychiatric symptoms.

Chronic obstructive pulmonary disease (COPD): LPS exposure, particularly in individuals who smoke or are exposed to air pollution, can exacerbate inflammation in the airways and lungs, contributing to the progression of COPD. LPS from inhaled pollutants or bacterial infections can activate immune cells in the lungs, leading to chronic inflammation, airway remodeling, and impaired lung function characteristic of COPD.

Chronic kidney disease (CKD): LPS has been implicated in the pathogenesis of CKD and its progression to end-stage renal disease (ESRD). In patients with CKD, impaired renal clearance of LPS can lead to systemic accumulation of endotoxin, contributing to chronic inflammation, oxidative stress, and progressive kidney damage. LPS-induced inflammation may also promote fibrosis and impair renal function in CKD.

Cholestasis: LPS has been implicated in the pathogenesis of cholestasis, a condition characterized by impaired bile flow from the liver to the intestine. LPS-induced inflammation in the liver can disrupt bile duct function and bile flow, leading to the accumulation of bile acids and bilirubin in the liver and bloodstream. This can result in jaundice, liver damage, and systemic complications. Cholestasis can occur in various conditions, including sepsis, liver infections, and drug-induced liver injury, where LPS-mediated inflammation may contribute to its development and progression.

Pulmonary hypertension: LPS-induced inflammation can contribute to the development of pulmonary hypertension, a condition characterized by elevated blood pressure in the pulmonary arteries. LPS-mediated activation of immune cells and release of pro-inflammatory cytokines can promote vasoconstriction, vascular remodeling, and endothelial dysfunction in the pulmonary vasculature, leading to increased pulmonary vascular resistance and right heart failure. This condition can occur in the context of acute lung injury, chronic lung diseases, or sepsis, where exposure to LPS may exacerbate pulmonary vascular pathology.

Acute pancreatitis: LPS has been implicated in the development and progression of acute pancreatitis, an inflammatory condition characterized by sudden inflammation of the pancreas. LPS can trigger an inflammatory response within the pancreas, leading to the activation of pancreatic enzymes and tissue damage. Additionally, LPS-induced systemic inflammation can contribute to the systemic complications of acute pancreatitis, such as sepsis and multiple organ failure.

Osteoarthritis: Emerging evidence suggests a potential link between LPS and the pathogenesis of osteoarthritis (OA). LPS can induce inflammation and cartilage degradation in the joints by activating immune cells and promoting the release of inflammatory mediators such as cytokines and matrix metalloproteinases. Chronic exposure to LPS, either from local sources within the joint or systemic circulation, may contribute to the progression of OA by exacerbating inflammation and tissue damage in the articular cartilage and synovium.

Cardiomyopathy: LPS-induced inflammation has been implicated in the development of cardiomyopathy, a group of diseases that affect the heart muscle. Chronic exposure to LPS can trigger inflammation in the myocardium, leading to cardiac dysfunction, fibrosis, and structural remodeling of the heart. LPS-mediated activation of immune cells and release of pro-inflammatory cytokines can contribute to myocardial injury and impaired cardiac function, ultimately leading to cardiomyopathy and heart failure.

Alcoholic liver disease (ALD): Chronic alcohol consumption can lead to increased intestinal permeability, allowing LPS to translocate from the gut into the bloodstream. Elevated levels of circulating LPS can trigger inflammation in the liver, contributing to the development and progression of alcoholic liver disease. LPS-mediated activation of immune cells in the liver leads to the release of pro-inflammatory cytokines, oxidative stress, and hepatocellular injury, ultimately resulting in liver inflammation, fibrosis, and cirrhosis. ALD encompasses a spectrum of liver disorders ranging from fatty liver (steatosis) to more severe forms such as alcoholic hepatitis and alcoholic cirrhosis, all of which can be exacerbated by LPS-induced inflammation.

Gastroesophageal reflux disease (GERD): LPS has been implicated in the pathogenesis of GERD, a chronic condition characterized by the reflux of stomach contents into the esophagus. LPS-induced inflammation in the gastrointestinal tract can disrupt the integrity of the esophageal mucosal barrier, making it more susceptible to damage from gastric acid and pepsin. Additionally, LPS-mediated activation of immune cells in the esophagus can lead to the release of pro-inflammatory cytokines, further exacerbating inflammation and tissue injury. Chronic exposure to LPS in the esophagus may contribute to the development and progression of GERD symptoms, such as heartburn, regurgitation, and esophageal mucosal damage.

Gastrointestinal cancers: Chronic exposure to LPS, particularly in the context of inflammatory conditions such as inflammatory bowel disease (IBD), has been implicated in the development of gastrointestinal cancers, including colorectal cancer and gastric cancer. LPS-induced inflammation can promote cellular proliferation, inhibit apoptosis, and induce DNA damage, contributing to the initiation and progression of cancerous lesions in the gastrointestinal tract. Additionally, LPS-mediated activation of immune cells and release of pro-inflammatory cytokines can create a tumor-promoting microenvironment, further supporting tumor growth and metastasis.

Autoimmune hepatitis (AIH): There is evidence suggesting that LPS may play a role in the pathogenesis of autoimmune hepatitis, a chronic inflammatory liver disease characterized by immune-mediated destruction of hepatocytes. LPS-induced activation of the innate immune system can trigger an inflammatory response in the liver, leading to the recruitment of immune cells and the release of pro-inflammatory cytokines. This immune dysregulation may contribute to the development and progression of autoimmune hepatitis, although the exact mechanisms are not fully understood. Additionally, elevated levels of circulating LPS have been observed in patients with autoimmune hepatitis, suggesting a potential link between LPS exposure and disease pathogenesis.

Preterm birth and neonatal complications: LPS exposure during pregnancy, particularly in cases of bacterial infection or inflammation, has been linked to preterm birth and neonatal complications. LPS can trigger an inflammatory response in the maternal-fetal interface, leading to premature labor and delivery. Additionally, LPS can cross the placental barrier and directly affect the developing fetus, increasing the risk of neonatal sepsis, respiratory distress syndrome, and other complications associated with prematurity. Infections during pregnancy, such as chorioamnionitis, can result in the release of LPS into the maternal bloodstream, further exacerbating the inflammatory response and increasing the risk of adverse pregnancy outcomes.

Asthma: LPS exposure has been implicated in the exacerbation and severity of asthma symptoms. Inhalation of airborne LPS, particularly in environments with high levels of endotoxin such as agricultural settings or homes with mold or dust, can trigger airway inflammation and bronchoconstriction in individuals with asthma. LPS-induced activation of immune cells in the airways leads to the release of pro-inflammatory cytokines and chemokines, recruitment of inflammatory cells, and airway hyperresponsiveness, contributing to asthma exacerbations and worsening of respiratory function. Individuals with asthma who are exposed to LPS may experience more frequent and severe asthma attacks, increased medication use, and reduced quality of life.

Necrotizing enterocolitis (NEC): LPS has been implicated in the development of necrotizing enterocolitis, a serious gastrointestinal condition primarily affecting premature infants. LPS-induced inflammation in the immature intestinal mucosa can lead to tissue damage, impaired barrier function, and necrosis of the bowel wall. Premature infants, with their immature gastrointestinal tract, are particularly vulnerable to the effects of LPS, which may be present in contaminated feeds or derived from bacterial colonization of the gut. NEC is characterized by abdominal distension, bloody stools, and signs of systemic illness, and it can lead to severe complications such as intestinal perforation and sepsis.

Non-alcoholic fatty liver disease (NAFLD): LPS has been implicated in the development and progression of NAFLD, a condition characterized by the accumulation of fat in the liver in individuals who do not consume excessive alcohol. LPS from the gut microbiota can translocate into the bloodstream due to increased intestinal permeability, leading to systemic inflammation and insulin resistance. LPS-induced inflammation in the liver can promote hepatic steatosis (fatty liver), inflammation, and fibrosis, contributing to the progression of NAFLD to more severe forms such as non-alcoholic steatohepatitis (NASH) and cirrhosis. Moreover, LPS-mediated activation of immune cells in the liver can exacerbate liver injury and promote the development of hepatocellular carcinoma (liver cancer) in patients with advanced NAFLD.

Chronic fatigue syndrome (CFS): Some studies suggest a potential link between LPS and chronic fatigue syndrome, a complex disorder characterized by persistent fatigue that is not alleviated by rest and is accompanied by other symptoms such as cognitive impairment, muscle pain, and sleep disturbances. LPS-induced inflammation may contribute to the pathophysiology of CFS, as immune dysregulation and chronic low-grade inflammation have been implicated in the development of this condition. Elevated levels of circulating LPS and increased intestinal permeability have been observed in some individuals with CFS, suggesting a possible role of LPS-mediated immune activation in the pathogenesis of the disease. However, more research is needed to fully elucidate the relationship between LPS and chronic fatigue syndrome.

Vascular dementia: There is emerging evidence suggesting a potential link between LPS and vascular dementia, a type of dementia characterized by cognitive impairment due to reduced blood flow to the brain. LPS-induced inflammation and endothelial dysfunction can contribute to the development and progression of vascular dementia by promoting atherosclerosis, cerebral small vessel disease, and microvascular dysfunction. Chronic exposure to LPS may exacerbate vascular pathology in the brain, leading to cognitive decline and vascular-related neurological deficits. Additionally, LPS-mediated activation of immune cells and release of inflammatory mediators in the brain can contribute to neuronal damage and white matter lesions, further impairing cognitive function in individuals with vascular dementia.

Gut-brain axis disorders: Emerging research suggests that LPS may play a role in the pathogenesis of gut-brain axis disorders, including irritable bowel syndrome (IBS), depression, and anxiety. LPS from the gut microbiota can translocate into the bloodstream due to increased intestinal permeability, leading to systemic inflammation and activation of the immune system. This systemic inflammation may contribute to the development of gut-brain axis disorders by affecting neuronal signaling, neurotransmitter function, and neuroinflammatory pathways. Additionally, LPS-induced alterations in the gut microbiota composition and function may further exacerbate gut-brain axis dysfunction, leading to the onset or exacerbation of symptoms in these conditions.

Autism spectrum disorder (ASD): Some studies suggest a potential link between maternal immune activation, including exposure to LPS, during pregnancy and an increased risk of ASD in offspring. LPS-induced activation of the maternal immune system can lead to the production of pro-inflammatory cytokines and chemokines, which may disrupt fetal brain development and contribute to the pathogenesis of ASD. Animal studies have demonstrated that prenatal exposure to LPS can result in behavioral abnormalities and neurodevelopmental deficits resembling those seen in ASD. While more research is needed to fully understand the relationship between LPS exposure and ASD, it is hypothesized that inflammatory processes triggered by LPS may play a role in the development of this complex neurodevelopmental disorder.

Obstructive sleep apnea (OSA): LPS has been implicated in the pathogenesis of obstructive sleep apnea, a common sleep disorder characterized by repetitive episodes of partial or complete obstruction of the upper airway during sleep. Chronic exposure to LPS, particularly in individuals with obesity or metabolic syndrome, can lead to inflammation and structural changes in the upper airway tissues, contributing to airway narrowing and collapsibility during sleep. LPS-induced inflammation in the upper airway mucosa can promote edema, fibrosis, and hypertrophy of the soft tissues surrounding the airway, further exacerbating airway obstruction and increasing the risk of OSA. Additionally, LPS-mediated activation of the innate immune system may contribute to the systemic inflammation and metabolic dysfunction observed in patients with OSA.

Interstitial cystitis/bladder pain syndrome (IC/BPS): Some research suggests a potential link between LPS and interstitial cystitis/bladder pain syndrome, a chronic inflammatory condition of the bladder characterized by pelvic pain, urinary urgency, and frequency. LPS-induced inflammation in the bladder epithelium and underlying tissues may contribute to the pathogenesis of IC/BPS by promoting bladder wall inflammation, epithelial barrier dysfunction, and neurogenic inflammation. Additionally, increased levels of circulating LPS and altered gut microbiota composition have been reported in patients with IC/BPS, suggesting a possible role of LPS-mediated immune activation and gut-bladder axis dysfunction in the development of this condition. However, further research is needed to fully elucidate the mechanisms underlying the association between LPS and IC/BPS.

Chronic rhinosinusitis (CRS): Some research suggests a potential link between LPS and chronic rhinosinusitis, a chronic inflammatory condition of the nasal and sinus mucosa characterized by nasal congestion, facial pain, and nasal discharge. LPS from environmental sources or bacterial colonization in the sinuses can trigger inflammation in the nasal and sinus epithelium, leading to mucosal edema, mucus production, and impaired mucociliary clearance. LPS-induced inflammation may also promote tissue remodeling and fibrosis in the nasal and sinus tissues, contributing to the development and persistence of CRS. Additionally, increased levels of LPS and alterations in the composition of the sinonasal microbiota have been observed in patients with CRS, suggesting a potential role of LPS-mediated immune activation and dysbiosis in the pathogenesis of this condition.

Amyotrophic lateral sclerosis (ALS): Emerging research suggests a potential link between LPS exposure and the development or progression of amyotrophic lateral sclerosis, a progressive neurodegenerative disease affecting motor neurons in the brain and spinal cord. LPS-induced inflammation and activation of microglia, the immune cells of the central nervous system, may contribute to the neuroinflammatory processes underlying ALS pathology. Chronic exposure to LPS may exacerbate neuroinflammation, neuronal damage, and motor neuron degeneration in individuals predisposed to ALS, although the exact mechanisms are not fully understood. Additionally, alterations in the gut microbiota composition and increased intestinal permeability, leading to higher levels of circulating LPS, have been reported in some patients with ALS, suggesting a potential role of LPS-mediated gut-brain axis dysfunction in the pathogenesis of this condition.

Interstitial Lung Disease (ILD): LPS exposure has been implicated in the development and progression of interstitial lung disease, a group of lung disorders characterized by inflammation and fibrosis of the lung parenchyma. Inhalation of LPS, particularly in occupational settings or in the context of environmental exposure, can trigger an inflammatory response in the lungs, leading to alveolar damage, fibroblast activation, and collagen deposition. LPS-induced inflammation and fibrosis contribute to the development of ILD and may worsen disease severity and progression. Additionally, increased levels of circulating LPS and alterations in the gut microbiota composition have been observed in patients with ILD, suggesting a potential role of LPS-mediated immune activation and dysbiosis in the pathogenesis of this condition.

Celiac disease: There is evidence suggesting that LPS may contribute to the pathogenesis of celiac disease, an autoimmune disorder characterized by an abnormal immune response to gluten, a protein found in wheat, barley, and rye. LPS from the gut microbiota or bacterial overgrowth in the small intestine can promote intestinal inflammation and barrier dysfunction, leading to increased permeability and translocation of gluten peptides and LPS into the bloodstream. This can trigger an immune response against gluten peptides and self-antigens in genetically susceptible individuals, resulting in the development of celiac disease. Additionally, LPS-induced inflammation in the gut mucosa may exacerbate tissue damage and perpetuate the autoimmune response in patients with celiac disease.

Autoimmune Encephalitis: Emerging research suggests a potential link between LPS and autoimmune encephalitis, a group of inflammatory disorders characterized by inflammation of the brain parenchyma due to autoantibodies targeting neuronal antigens. LPS-induced systemic inflammation and immune dysregulation may contribute to the breakdown of the blood-brain barrier and activation of autoimmunity in the central nervous system. This can lead to the production of autoantibodies against neuronal proteins, resulting in neuronal dysfunction, inflammation, and neurological symptoms. While the exact mechanisms linking LPS to autoimmune encephalitis are not fully understood, evidence suggests that LPS-mediated immune activation may play a role in the pathogenesis of this condition.

Gastric Ulcers: LPS has been implicated in the development of gastric ulcers, particularly in the context of Helicobacter pylori infection. LPS produced by H. pylori can trigger an inflammatory response in the gastric mucosa, leading to mucosal damage, erosion, and ulcer formation. Additionally, LPS-induced inflammation may disrupt the integrity of the gastric mucosal barrier, making it more susceptible to damage from gastric acid and other harmful factors. Chronic exposure to LPS from H. pylori infection can contribute to the development and exacerbation of gastric ulcers, which are characterized by abdominal pain, bleeding, and discomfort.

Idiopathic Pulmonary Fibrosis (IPF): LPS exposure has been implicated in the pathogenesis of idiopathic pulmonary fibrosis, a chronic and progressive lung disease characterized by the formation of scar tissue in the lungs. LPS can induce inflammation and fibrosis in the lung tissue by activating immune cells and promoting the release of pro-inflammatory and pro-fibrotic mediators. Chronic exposure to LPS, particularly in individuals with pre-existing lung conditions or environmental exposures, may contribute to the development and progression of idiopathic pulmonary fibrosis by exacerbating inflammation, promoting tissue remodeling, and impairing lung function. Additionally, LPS-mediated activation of the innate immune system may contribute to the dysregulated repair processes observed in idiopathic pulmonary fibrosis, leading to the accumulation of scar tissue and progressive decline in lung function over time.

Preeclampsia: There is evidence suggesting a potential link between LPS and preeclampsia, a pregnancy-related disorder characterized by high blood pressure and signs of organ damage, often affecting the kidneys and liver. LPS-induced inflammation and endothelial dysfunction may contribute to the pathogenesis of preeclampsia by promoting vasoconstriction, impaired placental perfusion, and oxidative stress. Chronic exposure to LPS, particularly in the context of bacterial infections or inflammatory conditions during pregnancy, may trigger an exaggerated immune response and endothelial dysfunction, leading to the development of preeclampsia. Additionally, alterations in the gut microbiota composition and increased intestinal permeability, which can result in higher levels of circulating LPS, have been reported in some women with preeclampsia, suggesting a potential role of LPS-mediated immune activation and dysbiosis in the pathogenesis of this condition.

Cardiovascular Diseases (CVD): There is growing evidence suggesting a link between LPS and cardiovascular diseases such as atherosclerosis, hypertension, and coronary artery disease. LPS can induce inflammation in the vascular endothelium, promoting the expression of adhesion molecules and the recruitment of immune cells to the vessel walls. This inflammatory response contributes to the development of atherosclerotic plaques and the progression of vascular damage. Additionally, LPS-induced inflammation can impair vascular function, leading to endothelial dysfunction and increased vascular permeability. Chronic exposure to LPS, particularly in conditions associated with systemic inflammation such as obesity, metabolic syndrome, and periodontal disease, may increase the risk of cardiovascular events and contribute to the pathogenesis of cardiovascular diseases.

Polycystic Ovary Syndrome (PCOS): Emerging research suggests a potential link between LPS and polycystic ovary syndrome, a hormonal disorder characterized by irregular menstrual periods, excess androgen levels, and ovarian cysts. LPS-induced inflammation may contribute to the pathogenesis of PCOS by promoting insulin resistance, hyperandrogenism, and ovarian dysfunction. Chronic exposure to LPS, particularly in individuals with obesity or metabolic syndrome, may exacerbate inflammation and metabolic disturbances associated with PCOS, leading to the development or worsening of symptoms. Additionally, alterations in the gut microbiota composition and increased intestinal permeability, which can result in higher levels of circulating LPS, have been reported in some women with PCOS, suggesting a potential role of LPS-mediated gut dysbiosis and immune activation in the pathogenesis of this condition.

Endometriosis: Emerging evidence suggests a potential link between LPS and endometriosis, a chronic inflammatory condition characterized by the presence of endometrial-like tissue outside the uterus, leading to pelvic pain and infertility. LPS-induced inflammation may contribute to the pathogenesis of endometriosis by promoting the growth and survival of ectopic endometrial tissue, as well as by stimulating the production of pro-inflammatory cytokines and chemokines. Chronic exposure to LPS, particularly in the context of bacterial infections or pelvic inflammatory conditions, may exacerbate inflammation and tissue damage associated with endometriosis. Additionally, alterations in the gut microbiota composition and increased intestinal permeability, which can result in higher levels of circulating LPS, have been reported in some women with endometriosis, suggesting a potential role of LPS-mediated gut dysbiosis and immune activation in the pathogenesis of this condition.

Periodontal Disease: There is evidence suggesting a potential link between LPS and periodontal disease, a chronic inflammatory condition affecting the tissues surrounding the teeth. LPS is a major component of the cell wall of Gram-negative bacteria commonly found in dental plaque. When these bacteria accumulate along the gum line, LPS can be released and trigger an immune response in the gingival tissues. This immune response leads to inflammation, tissue destruction, and bone loss characteristic of periodontal disease. Additionally, systemic inflammation resulting from chronic periodontal infection, including elevated levels of circulating LPS, has been associated with an increased risk of cardiovascular disease, diabetes, and other systemic conditions. Therefore, LPS may play a role in the pathogenesis and progression of periodontal disease as well as its systemic implications.

Accordingly, in some embodiments, administering an effective amount of one or more peptoid compounds to a subject may treat or prevent one or more of the pathological conditions described herein.

In some embodiments, the one or more peptoid compounds may prevent or decrease activity of LPS in the subject.

In some embodiments, the activity of LPS may be decreased by up to 10%, 20%, 30%, 40%, 50%, 60%, 70%, 80%, 90%, 95%, 99%, or more.

In some embodiments, the peptoid compounds can bind LPS and neutralize, decrease or inhibit the effects of LPS.

In some embodiments, the peptoid compounds can prevent, increase, or decrease binding of the LPS to one or more LPS-binding proteins in a subject.

LPS-binding proteins are a group of proteins that play important roles in the recognition, binding, and immune response to LPS. LPS-binding proteins are involved in various physiological processes, including host defense, inflammation, and regulation of the immune response. For example, in some embodiment, the LPS-binding proteins may include, without limitation, the following: LPS-binding protein (LBP): LBP is a soluble acute-phase protein produced primarily by hepatocytes in response to infection and inflammation. It binds to LPS and facilitates its transfer to CD14 and Toll-like receptor 4 (TLR4), thereby initiating the immune response to bacterial infection.

CD14: CD14 is a glycosylphosphatidylinositol (GPI)-anchored protein found on the surface of monocytes, macrophages, and neutrophils. It acts as a co-receptor for TLR4 and facilitates the recognition of LPS by the TLR4/MD-2 complex, leading to the activation of downstream signaling pathways.

MD-2: MD-2 is a co-receptor for TLR4 that forms a complex with TLR4 and binds directly to LPS. It is required for the activation of TLR4 signaling in response to LPS.

Toll-like receptor 4 (TLR4): TLR4 is a transmembrane receptor expressed on immune cells, including macrophages, dendritic cells, and B cells. It recognizes LPS in conjunction with MD-2 and initiates intracellular signaling pathways leading to the production of pro-inflammatory cytokines and chemokines.

Soluble CD14 (sCD14): sCD14 is a truncated form of CD14 that lacks the GPI anchor and is released into the circulation. It acts as a decoy receptor for LPS, competing with membrane-bound CD14 for LPS binding and modulating the immune response to LPS.

Lipopolysaccharide and beta-1,3-glucan binding protein (LGBP): LGBP is an LPS-binding protein found in invertebrates, including insects and crustaceans. It plays a role in the recognition and clearance of bacterial pathogens by the innate immune system.

Pentraxins: Pentraxins, such as C-reactive protein (CRP) and serum amyloid P component (SAP), can bind to LPS and modulate the immune response to bacterial infection. They are involved in opsonization, complement activation, and clearance of pathogens.

Surfactant proteins: Surfactant proteins, including surfactant protein A (SP-A) and surfactant protein D (SP-D), are found in pulmonary surfactant and can bind to LPS, contributing to host defense in the lungs.

Lipopolysaccharide-binding protein 2 (LBP-2): LBP-2 is another soluble acute-phase protein similar to LBP. It is involved in the recognition and binding of LPS and may contribute to the activation of the immune response to bacterial infection.

Bactericidal/permeability-increasing protein (BPI): BPI is an antimicrobial protein found in neutrophil granules. It binds to and neutralizes the toxic effects of LPS, including its ability to induce cytokine production and activate the coagulation cascade.

Limulus anti-LPS factor (LALF): LALF is a protein found in the hemolymph of horseshoe crabs (Limulus polyphemus). It binds to and neutralizes LPS, protecting the crab from bacterial infection.

Bacterial LPS-binding protein (BLBP): BLBP is a protein produced by certain bacteria that binds to LPS and may play a role in bacterial virulence or host-pathogen interactions.

Plasma lipopolysaccharide-binding protein (PLBP): PLBP is a protein found in the plasma of some animals that binds to LPS and may contribute to the clearance of LPS from the circulation.

Ficolins: Ficolins are a family of pattern recognition receptors that can bind to carbohydrates, including LPS. They are involved in the activation of the lectin pathway of complement and the clearance of pathogens.

Mannose-binding lectin (MBL): MBL is a pattern recognition receptor that can bind to carbohydrates, including LPS. It plays a role in the activation of the lectin pathway of complement and the opsonization of pathogens.

Lymphocyte antigen 96 (LY96): LY96, also known as myeloid differentiation factor 2 (MD-2)-related protein (MD-2RP), is a protein that interacts with MD-2 and may play a role in LPS recognition and signaling.

Cationic Antimicrobial Peptides (CAMPs): Certain cationic antimicrobial peptides, such as cathelicidins and defensins, have been shown to bind to and neutralize LPS. These peptides contribute to the innate immune response by disrupting bacterial membranes and promoting clearance of pathogens.

Periplasmic Binding Proteins (PBPs): In bacteria, periplasmic binding proteins are involved in the uptake of nutrients and other molecules from the environment. Some periplasmic binding proteins have been shown to bind to LPS, potentially contributing to the transport of LPS into the bacterial cell.

Lipopolysaccharide Transport Proteins: Various proteins involved in the biosynthesis and transport of LPS within Gram-negative bacteria may also bind to LPS. These proteins include enzymes involved in LPS biosynthesis and assembly, as well as transporters responsible for the export of LPS to the outer membrane.

Scavenger Receptors: Scavenger receptors are cell surface receptors that bind to a variety of ligands, including modified lipoproteins and bacterial products such as LPS. Some scavenger receptors have been implicated in the recognition and clearance of LPS from the circulation.

Lipid-Binding Proteins: Certain lipid-binding proteins may also interact with LPS due to their ability to bind to lipid molecules. Examples include fatty acid-binding proteins and other intracellular lipid chaperones.

Tumor Necrosis Factor Receptor Superfamily Member 6 (TNFRSF6): TNFRSF6, also known as FAS or CD95, has been reported to interact with LPS and modulate cellular responses to LPS stimulation.

LPS-binding activity of peptoid compounds may be identified, confirmed and/or quantified using methods such as those described in Example 2 of the present disclosure.

The terms “peptoid” or “peptoid compound” as used herein refers to a type of biomimetic molecule that is similar to peptides but differs in its structure. Peptoids are synthetic oligomers composed of N-substituted glycine units. Accordingly, peptoids are also known as poly-N-substituted glycine compounds. In contrast to peptides, which have a peptide bond between amino acids, peptoids have a N-substituted (or N-alkylated) amide bond. This structural difference gives peptoids unique properties compared to peptides. Peptoids can be designed and synthesized to mimic the functions of natural peptides but with enhanced stability and different chemical properties. Peptoid compounds may be cyclic or linear. Peptoids have been described, for example, in U.S. Pat. Nos. 8,445,632, 8,828,413, 9,315,548, 9,872,495, 9,938,321, and International Patent Application Publication No.'s WO2021046562, WO2020223581, WO2021127294, WO2023287570, WO2022120393, and WO2021231343, the disclosures of which are incorporated herein in their entireties.

For example, without limitation, in some embodiments a peptoid compound may have a formula:

A [symbol] X—Y—Z [symbol] ₙB.

In such a compound, A can be selected from H and a terminal N-alkyl substituted glycine residue, where such an alkyl substituent can be selected from about C4 to about C20 linear, branched and cyclic alkyl moieties; n can be an integer selected from 1-3; B can be selected from NH2, and one and two N-substituted glycine residues, such N-substituents as can be independently selected from α-amino acid side chain moieties and structural/functional analogs thereof; and X, Y and Z can also be independently selected from N-substituted glycine residues, such N-substituents as can be independently selected from α-amino acid side chain moieties and structural/functional analogs thereof and proline residues. Such X—Y—Z periodicity can provide such a compound a certain amphipathicity. As would be understood by those skilled in the art, such structural and/or functional analogy can be considered in the context of any such α-amino acid side chain, N-substituent and/or a sequence of such N-substituted glycine residues, such structure and/or function including but not limited to charge, chirality, hydrophobicity, amphipathicity, helical structure and facial organization. Such analogs include, without limitation, carbon homologs of such side chain-such homologs as would be understood in the art, including but not limited to plus or minus 1 or 2 or more methylene and/or methyl groups.

A can be H, and B can be selected from one or two N-substituted glycine residues, such a selection as can reduce the hydrophobicity of such a compound, as compared to compounds of 3-fold periodicity. In certain such embodiments, X can be an NLys residue; n can be 2-3; and B can be two N-substituted glycine residues. Without limitation, such a compound can be of a formula:

H [symbol] N_{Lys}—Nₛₚₑ—Nₛₚₑ [symbol] ₃N_{Lys}—Nₛₚₑ—NH₂.

Regardless of identity of A, X and B, at least one of Y and Z can be a proline residue. X, Y and Z can be proline residues.

In certain other embodiments, A can be a terminal N-alkyl substituted glycine residue, with such an alkyl substituent as can be selected from about C6 to about C18 linear alkyl moieties.

Regardless, B can be NH2, and n can be selected from 1 and 2. In certain such embodiments, A can be a terminal N-alkyl substituted glycine residue, with an alkyl substituent selected from about C6 to about C18 linear alkyl moieties. Regardless, B can be an NLys residue, and n can be 1.

In some embodiments, a peptoid compound may have a formula:

H [symbol] N_{Lys}—Y—Z [symbol] ₙY′—Z′—NH₂
wherein n can be selected from 2 and 3; and Y, Z, Y′ and Z′ can be independently selected from N-substituted glycine residues, where such substituents can be independently selected from α-amino acid side chain moieties and carbon homologs thereof. Such Y′ and Z′ residues can be selected to provide such compound reduced hydrophobicity as compared to a compound of 3-fold periodicity. In certain such embodiments, at least one of X and Y can be a proline residue. Regardless, n can be selected from 2 and 3, and Y′ can be an N_{Lys} residue. In certain such embodiments, one or both X and Y can be proline residues. Without limitation, such a compound with reduced hydrophobicity can be of a formula:

H [symbol] N_{Lys}—Nₛₚₑ—Nₛₚₑ [symbol] ₃N_{Lys}—Nₛₚₑ—NH₂

In some embodiments, a peptoid compound may have a formula:

H—N_{R} [symbol] X—Y—Z [symbol] ₙ B
wherein B can be selected from NH₂ and X′; X, Y, Z and X can be independently selected from N-substituted glycine residues, where such substituents can be independently selected from α-amino acid side chain moieties and carbon homologs thereof; n can be an integer selected from 1 and 2; and R can be an N-alkyl substituent of such a glycine residue, as can be selected from about C₄ to about C₂₀ linear, branched and cyclic alkyl moieties. In some embodiments, n can be 2, and B can be NH₂. In some embodiments, n can be 1, and B can be X′. Accordingly, one or both of X and X′ can be N_{Lys} residues. Regardless, an alkyl substituent can be selected from about C₆ to about C₁₈ linear, branched and cyclic alkyl moieties, and X and X′ can be N_{Lys} residues. Without limitation, such a compound can be of a formula:

H—N_{tridec}—N_{Lys}—Nₛₚₑ—Nₛₚₑ—N_{Lys}—NH₂.

A peptoid may be a poly-N-substituted glycine compound comprising an N-terminus selected from H and an N-alkyl substituted glycine residue, where such an alkyl substituent can be selected from about C₄ to about C20 linear, branched and cyclic alkyl moieties; a C-terminus selected from NH₂, one and two N-substituted glycine residues, such N-substituents as can be independently selected from α-amino acid side chain moieties and structural/functional analogs thereof; and 2 to about 15 monomeric residues between the N- and C-termini, each such residue as can be independently selected from proline residues and N-substituted glycine residues, said N-substituents independently selected from α-amino acid side chain moieties and structural/functional analogs thereof. Such monomers can be selected to provide such a compound a non-periodic sequence of monomers. As would be understood by those skilled in the art, such structural and/or functional analogy can be considered in the context of any such α-amino acid side chain, N-substituent and/or a sequence of such N-substituted glycine residues, such structure and/or function including but not limited to charge, chirality, hydrophobicity, amphipathicity, helical structure and facial organization. Such analogs include, without limitation, carbon homologs of such side chain-such homologs as would be understood by those skilled in the art, including but not limited to plus or minus 1 or 2 or more methylene and/or methyl groups.

The N-terminus of such a compound can be H; and the C-terminus can be selected from said one and two N-substituted glycine residues. A peptoid compound can comprise 2 to about 5 (X—Y—Z) non-periodic trimers. At least one of X, Y and Z in each of the trimers can be selected to interrupt 3-fold periodicity. Without limitation, at least one X in at least one said trimer can be an NLys residue. At least one of Y and Z in at least one such trimer can be a proline residue. The monomeric residues can comprise at least two non-consecutive of the same or repeat trimers, with at least one such residue therebetween to interrupt periodicity. At least one X in at least one such trimer can be an NLys residue, and at least one of Y and Z in at least one said trimer can be a proline residue.

The N-terminus of such a compound can be an N-alkyl substituted glycine residue, with an alkyl substituent selected from about C6 to about C18 linear alkyl moieties. A peptoid compound can comprise 2 to about 5 (X—Y—Z) non-periodic trimers. At least one of X, Y and Z in each of the trimers can be selected to interrupt 3-fold periodicity. The monomeric residues can comprise at least two non-consecutive of the same or repeat trimers, with at least one residue therebetween to interrupt periodicity. At least one X in at least one said trimer can be an NLys residue, and at least one of Y and Z in at least one said trimer can be a proline residue.

Various halogenated peptoids may be utilized in accordance with the teachings herein to make antiviral pharmaceutical compositions and treatments. These include, without limitation, various halogenated analogs of the foregoing peptoid compounds. These halogenated compositions may be halogenated in various ways. For example, these compounds may include any number of halogen substitutions with the same or different halogens. In particular, these compounds may include one or more fluoro-, chloro-, bromo- or iodo-substitutions, and may include substitution with two or more distinct halogens. In some embodiments, the use of one or two bromo- or chloro-substitutions may be used. The peptoids described herein may be halogenated at various locations, for example and without limitation para halogenation on the peptoids containing aryl rings, ortho- and meta-substitution, or perhalogentation.

The peptoids described herein may be alkylated, for example and without limitation terminal alkylation. For example and without limitation, the alkyl substituent may be selected from about C₆ to about C₁₈ linear alkyl moieties.

In some embodiments, a peptoid may have antibacterial activity, antifungal activity, antiviral activity, or any combination thereof.

Without intending to be bound by theory, the peptoid compounds described herein mimic the structures and functions of antimicrobial peptides, key constituents of the human immune system, to exert broad direct antibacterial, antiviral and antifungal activity. Peptoids are structural variants of peptides, in which the side chain groups are appended to nitrogen (instead of carbon) to form an amphiphilic molecule with both hydrophobic and cationic features. This novel structure resists proteolysis to form a more stable compound in vivo with the same anti-pathogenic properties as natural peptides.

Without intending to be bound by theory, antiviral activity of a peptoid may be associated with its ability to pass through a viral membrane and to bind to viral DNA or RNA. Furthermore, also without intending to be bound by theory, the mechanism of action may also feature disruption of membranes of various pathogens, by preferentially interacting with the lipid phosphatidylserine, which is found on the outer leaflet of various pathogen membranes. Phosphatidylserine is not typically present on mammalian cell surfaces, allowing peptoid compounds to exhibit selectivity towards microbial cell types. The peptoid compounds described herein offer substantial pharmacological advantages over monoclonal antibodies and biological therapeutics: smaller size, low risk of off target effects, low manufacturing cost, anti-inflammatory properties, no cold chain requirement, high stability in vivo, and multiple mechanisms of action.

Various peptoid compounds may be utilized in accordance with the teachings herein to make pharmaceutical compositions and treatments, including without limitation the peptoid compounds described in the various patents and patent application publications described herein, which are incorporated herein in their entireties.

The peptoids described herein may be synthesized and provided by any suitable method known in the art, such as, for example and not by way of limitation, the method described in Example 1 of the present disclosure, or by methods described in the patents and patent application publications disclosed herein.

Various counterions may be utilized in forming pharmaceutically acceptable salts of the peptoids disclosed herein. In some embodiments, pharmaceutically acceptable salts of the peptoids disclosed herein may include sodium or hydrochloride salts.

In some embodiments, the present disclosure extends to the preparation of prodrugs and derivatives of the peptoids of the invention. Prodrugs are derivatives which have cleavable groups and become by solvolysis or under physiological conditions the peptoid of the invention, which are pharmaceutically active. Such examples include, but are not limited to, choline ester derivatives and the like, N-alkylmorpholine esters and the like. In some embodiments, the peptoid compounds provided herein may be prepared e.g., in crystalline form and may be solvated or hydrated. Suitable solvates include pharmaceutically acceptable solvates, such as hydrates, and further include both stoichiometric solvates and non-stoichiometric solvates.

In some embodiments, the one or more of the peptoid compounds described herein may be used in the methods and compositions described herein.

In some embodiments, the one or more peptoid compounds may be formulated in a composition comprising the one or more peptoid compounds and one or more pharmaceutically acceptable excipients.

In some embodiments, the composition may be formulated for administration including but not limited to, topical administration, transdermal administration, transmucosal administration, intraperitoneal administration, subcutaneous administration, intramuscular administration, or intravenous administration to the subject.

Such compositions can be prepared in a manner known in the pharmaceutical art. The peptoid compounds described herein can be formulated into pharmaceutically acceptable compositions and dosage forms for administration to a subject. In some embodiments, the present disclosure relates to a composition comprising an effective amount of a peptoid compound described herein for use in a method of treating a subject. In some embodiments, the present disclosure relates to the use of the peptoids described herein for the preparation of medicaments or as medicaments, that may be used in the methods descried herein.

The present disclosure provides pharmaceutical compositions comprising one or more peptoids and a pharmaceutically acceptable medium, such as an excipient, carrier, or the like. The peptoids described herein may be dissolved, suspended or disposed in various media. Such media may include, for example, various liquid, solid or multistate media such as, for example, emulsions, gels or creams. Such media may include liquid media, which may be hydrophobic or may comprise one or more triglycerides or oils. Such media may include, but is not limited to, vegetable oils, fish oils, animal fats, hydrogenated vegetable oils, partially hydrogenated vegetable oils, synthetic triglycerides, modified triglycerides, fractionated triglycerides, and mixtures thereof. Triglycerides used in these pharmaceutical compositions may include those selected from the group consisting of almond oil; babassu oil; borage oil; blackcurrant seed oil; black seed oil; canola oil; castor oil; coconut oil; corn oil; cottonseed oil; evening primrose oil; grapeseed oil; groundnut oil; mustard seed oil; olive oil; palm oil; palm kernel oil; peanut oil; rapeseed oil; safflower oil; sesame oil; shark liver oil; soybean oil; sunflower oil; hydrogenated castor oil; hydrogenated coconut oil; hydrogenated palm oil; hydrogenated soybean oil; hydrogenated vegetable oil; hydrogenated cottonseed and castor oil; partially hydrogenated soybean oil; soy oil; glyceryl tricaproate; glyceryl tricaprylate; glyceryl tricaprate; glyceryl triundecanoate; glyceryl trilaurate; glyceryl trioleate; glyceryl trilinoleate; glyceryl trilinolenate; glyceryl tricaprylate/caprate; glyceryl tricaprylate/caprate/laurate; glyceryl tricaprylate/caprate/linoleate; glyceryl tricaprylate/caprate/stearate; saturated polyglycolized glycerides; linoleic glycerides; caprylic/capric glycerides; modified triglycerides; fractionated triglycerides; and mixtures thereof.

Various fatty acids may be utilized in the pharmaceutical compositions disclosed herein. These include, without limitation, both long and short chain fatty acids. Examples of such fatty acids include, but are not limited to, docosahexaenoic acid, caprylic acid, capric acid, lauric acid, butyric acid, and pharmaceutically acceptable salts thereof.

Generally, the peptoid compounds described herein are administered in a therapeutically effective amount. “Therapeutically effective amount” means the amount of a compound that, when administered to a subject for treating a disease, is sufficient to effect such treatment. The therapeutically effective amount of the peptoid compound may be determined by a physician, in the light of the relevant circumstances, including the condition to be treated, the chosen route of administration, the peptoid compound administered, the age, weight, and response of the individual subject, the severity of the subject's symptoms, and the like.

In some embodiments, the effective amount may be from 1-1000 mg/day, 25-750 mg/day, 50-500 mg/day, or 100-400 mg/day.

Moreover, these compositions may be administered in a single dose, multi-dose or controlled release fashion.

The term “administering”, “administered” and grammatical variants refers to the physical introduction of an agent to a subject, using any of the various methods and delivery systems known to those skilled in the art. Exemplary routes of administration include intravenous, intramuscular, subcutaneous, intraperitoneal, spinal or other parenteral routes of administration, for example by injection or infusion. The phrase “parenteral administration” means modes of administration other than enteral and topical administration, usually by injection, and includes, without limitation, intravenous, intramuscular, intraarterial, intrathecal, intralymphatic, intralesional, intracapsular, intraorbital, intraocular, intracardiac, intradermal, intraperitoneal, transtracheal, subcutaneous, subcuticular, intraarticular, subcapsular, subarachnoid, intraspinal, epidural and intrasternal injection and infusion, as well as in vivo electroporation. Non-parenteral routes include oral, topical, epidermal or mucosal route of administration, for example, intranasally, vaginally, rectally, sublingually or topically. Administering can also be performed, for example, once, a plurality of times, and/or over one or more extended periods.

In some embodiments, the administration may be one, two, three, or four times per day. In some embodiments, the administration may be once per week, once every two weeks, or once per month.

Formulation for topical administration may include, for example, dry powder formulation with a polymer to potentially extend residence time and drug release rate, spray on foam, topical gel, and an aqueous solution. These formulations can be dressed with a bandage or hemostatic gauze to maintain the formulation in place on the skin and to provide a protective barrier. A powder formulation containing drug and polymer may be provided in a sachet or stick pack, where it could be administered directly to the skin or suspended in an aqueous solution for irrigation and administration. A simple powder formulation may be dissolved in an aqueous solution for irrigation and administration. A spray on foam formulation or a gel formulation may be administered via a small aerosol container. These product types provide for variable dosing and are suited for unpredictable conditions, i.e. windy, wet, varied temperatures, or may be used in a field hospital or tertiary care facility.

The pharmaceutical compositions disclosed herein may be manufactured as tablets, liquids, gels, foams, ointments or powders. In some embodiments, these compositions may be applied as microparticles or nanoparticles.

In some embodiments, intranasal compositions may comprise any pharmaceutically acceptable excipient, such as those approved in nasal spray formulations and listed in the Food and Drug Administration's Inactive Ingredient Database, or justifiable based on the Food and Drug Administration's Guidance for Industry: Nasal Spray and inhalation Solution, Suspension, and Spray Drug Products—Chemistry, manufacturing, and Controls Documentation. As would be understood by skilled persons, typically, the excipients used in intranasal formulations should be safe and compatible with nasal mucosa. Some common excipients used in intranasal products include buffers to maintain the pH of the formulation within an acceptable range, preservatives to prevent microbial contamination, surfactants to enhance drug absorption and distribution, stabilizers to maintain the stability of the formulation over time, solubilizers to improve the solubility of poorly soluble drugs, viscosity modifiers to control the viscosity of the formulation for better administration, and tonicity agents to adjust the osmolarity of the formulation to be close to that of nasal mucosa.

Compositions for oral administration can take the form of bulk liquid solutions or suspensions, or bulk powders. More commonly, however, the compositions are presented in unit dosage forms to facilitate accurate dosing. The term “unit dosage forms” refers to physically discrete units suitable as unitary dosages for subjects, each unit containing a predetermined quantity of active material calculated to produce the desired therapeutic effect, in association with a suitable pharmaceutical excipient. Typical unit dosage forms include prefilled, premeasured ampoules or syringes of the liquid compositions or pills, tablets, capsules or the like in the case of solid compositions. In such compositions, the peptoid compound is usually a minor component (e.g., from about 0.01% to about 50% by weight) with the remainder being various vehicles or carriers and processing aids helpful for forming the desired dosing form.

Liquid forms suitable for oral administration may include, without limitation, a suitable aqueous or nonaqueous vehicle with buffers, suspending and dispensing agents, colorants, flavors and the like.

Solid forms may include, without limitation any of the following ingredients, or compounds of a similar nature: a binder such as microcrystalline cellulose, gum tragacanth or gelatin; an excipient such as starch or lactose, a disintegrating agent such as alginic acid, Primogel, or cornstarch; a lubricant such as magnesium stearate; a glidant such as colloidal silicon dioxide; a sweetening agent such as sucrose or saccharin; or a flavoring agent such as peppermint, methyl salicylate, or orange flavoring.

Injectable compositions are typically based upon injectable sterile saline or phosphate-buffered saline or other injectable carriers known in the art. The peptoid compound in such compositions is typically a minor component, often being from about 0.05% to 10% by weight with the remainder being the injectable carrier and the like.

Transdermal compositions are typically formulated as a topical ointment or cream containing the peptoid compound, generally in an amount ranging from about 0.01 to about 20% by weight. When formulated as an ointment, the peptoid compound may be combined with either a paraffinic or a water-miscible ointment base. The peptoid compound may be formulated in a cream with, for example an oil-in-water cream base. Such transdermal formulations may include additional ingredients to enhance the dermal penetration of stability of the peptoid compounds or the formulation.

The peptoid compounds of the present disclosure can be administered by a transdermal device. Accordingly, transdermal administration can be accomplished using a patch either of the reservoir or porous membrane type, or of a solid matrix variety.

The peptoid compounds of the present disclosure can be administered subcutaneously, including, without limitation, the use of syringe and needle injection, autoinjectors, pen injectors, needle-free injectors, subcutaneous infusion, jet injectors, patch pumps, pump infusion sets, implantable devices, subcutaneous depots, subcutaneous sustained release formulations, or any combinations thereof. The most traditional and widely used method of subcutaneous administration involves using a syringe and needle. Autoinjectors are pre-filled devices that automatically inject a set dose of a pharmaceutical composition when pressed against the skin. Examples include, without limitation, EpiPen for epinephrine and various biologic medications. Similar to autoinjectors, pen injectors are pre-filled devices that allow patients to self-administer a specific dose of a pharmaceutical composition. They are user-friendly and may have features like dose adjustment. Needle-free injectors use high pressure to administer a pharmaceutical composition through the skin without using a needle. Subcutaneous infusion may involve using an infusion pump to deliver a continuous or intermittent flow of a pharmaceutical composition into the subcutaneous tissue. Jet injectors use a high-pressure stream of liquid to penetrate the skin and deliver a pharmaceutical composition into the subcutaneous tissue. Patch pumps may adhere to the skin and contain a reservoir of a pharmaceutical composition, and it is absorbed through the skin over a period of time. Pump infusion sets may include a cannula or needle that is placed under the skin for continuous pharmaceutical composition delivery. Implantable devices may be used to provide sustained release of a pharmaceutical composition subcutaneously. Implantable devices may be surgically implanted and can deliver a controlled dose of a pharmaceutical composition over an extended period.

Subcutaneous depot release refers to the administration of pharmaceutical compositions in a way that allows for sustained and controlled release of a pharmaceutical composition from a depot or reservoir located in the subcutaneous tissue. This method may be used to provide a prolonged therapeutic effect, reducing the frequency of dosing and improving patient compliance. In some embodiments, a subcutaneous depot release formulation may include, without limitation, a solution, a suspension, or biodegradable matrix, that is introduced (e.g., injected) into the subcutaneous tissue. The formulation then forms a depot, a localized reservoir of a pharmaceutical composition, beneath the skin. The subcutaneous depot formulation may release an active substance, e.g. a peptoid, gradually over an extended period.

A subcutaneous depot formulation may include, without limitation, biodegradable matrices, liposomal formulations, polymeric microspheres or nanoparticles, hydrogels, PLGA (poly(lactic-co-glycolic acid)) microparticles, implantable devices, or any combinations thereof. In biodegradable polymers or matrices, over time, the matrix breaks down, releasing a pharmaceutical composition in a controlled manner. Liposomes, which are lipid vesicles, can encapsulate a pharmaceutical composition and provide controlled release. Such liposomal formulations may be injected subcutaneously to create a depot of a pharmaceutical composition. Microspheres or nanoparticles made of biocompatible polymers can encapsulate a pharmaceutical composition and release it slowly over time. These particles can be suspended in a liquid formulation and injected into the subcutaneous tissue. Hydrogels are water-containing gels that can hold and release a pharmaceutical composition. Injectable hydrogels can form depots in the subcutaneous tissue. PLGA microparticles comprise PLGA, a biodegradable polymer commonly used to create microparticles for sustained drug release. PLGA microparticles can be injected subcutaneously to form a depot. Some subcutaneous depot release systems involve implantable devices, such as osmotic pumps or reservoirs. These devices are typically placed under the skin during a minor surgical procedure and provide controlled release of a pharmaceutical composition for an extended period.

Example formulations and methods of sustained release subcutaneous administration of the peptoids and pharmaceutical compositions thereof described herein include those described in the following references, the contents of all of which are incorporated herein in their entireties: Judy Senior, Michael L. Radomsky. (2000). Sustained-Release Injectable Products. Boca Raton: CRC Press; Thambi T, Li Y, Lee D S. Injectable hydrogels for sustained release of therapeutic agents. J Control Release. 2017 Dec. 10; 267:57-66. doi: 10.1016/j.jconrel.2017.08.006. Epub 2017 Aug. 4. PMID: 28827094.; Chan Y P, Meyrueix R, Kravtzoff R, Nicolas F, Lundstrom K. Review on Medusa: a polymer-based sustained release technology for protein and peptide drugs. Expert Opin Drug Deliv. 2007 July; 4(4):441-51. doi: 10.1517/17425247.4.4.441. PMID: 17683256.; Lou H, Feng M, Hageman M J. Advanced Formulations/Drug Delivery Systems for Subcutaneous Delivery of Protein-Based Biotherapeutics. J Pharm Sci. 2022 November; 111(11):2968-2982. doi: 10.1016/j.xphs.2022.08.036. Epub 2022 Sep. 2. PMID: 36058255; Sequeira J A D, Santos A C, Serra J, Estevens C, Seiga R, Veiga F, Ribeiro A J. Subcutaneous delivery of biotherapeutics: challenges at the injection site. Expert Opin Drug Deliv. 2019 February; 16(2):143-151. doi: 10.1080/17425247.2019.1568408. Epub 2019 Jan. 24. PMID: 30632401; Badkar A V, Gandhi R B, Davis S P, LaBarre M J. Subcutaneous Delivery of High-Dose/Volume Biologics: Current Status and Prospect for Future Advancements. Drug Des Devel Ther. 2021 Jan. 13; 15:159-170. doi: 10.2147/DDDT.S287323. PMID: 33469268; PMCID: PMC7812053; Vaishya R, Khurana V, Patel S, Mitra A K. Long-term delivery of protein therapeutics. Expert Opin Drug Deliv. 2015 March; 12(3):415-40. doi: 10.1517/17425247.2015.961420. Epub 2014 Sep. 24. PMID: 25251334; PMCID: PMC4605535; Remington's Pharmaceutical Sciences, 17th edition, 1985, Mack Publishing Company, Easton, Pa. The above-described components for intranasal, orally administrable, injectable subcutaneous, or topically administrable compositions are merely representative. Other materials as well as processing techniques and the like that are suitable for administering the peptoids and pharmaceutical compositions described herein are identifiable by skilled persons upon reading the present disclosure.

The peptoid compounds described herein can be administered in sustained release forms or from sustained release or controlled drug delivery systems, delivered via oral, intramuscular, subcutaneous, or transdermal route. A description of representative sustained release materials and description of delivery systems can be found in Remington's Pharmaceutical Sciences and Modern Pharmaceutics.

In some embodiments, the formulations described herein may include one or more chelation agents. In some embodiments, the chelation agent may be an efficacious anti-calculus agent including, but not limited to, one or more of zinc, hexametaphosphates, and diphosphonates.

In some embodiments, the formulations described herein may include one or more chelation agents selected from aminopolycarboxylic acids, citric acid, edetate disodium anhydrous, edetate calcium disodium anhydrous citrate salts, sodium gluconate, transferrins, polymers, and any combinations thereof. In some embodiments, the aminopolycarboxylic acids may be selected from the group consisting of tetraxetan (DOTA), nitrilotriacetic acid (NTA), Ethylenediaminetetraacetic acid (EDTA or EDTA acid), ethylene glycol-bis(β-aminoethyl ether)-N,N,N′,N′-tetraacetic acid (EGTA or egtazic acid), 1,2-bis(o-aminophenoxy)ethane-N,N,N′,N′-tetraacetic acid (BAPTA), pentetic acid, diethylenetriaminepentaacetic acid (DTPA) nicotianamine, ethylenediamine-N,N′-bis(2 hydroxyphenylacetic acid) (EDDHA), Ethylenediamine-N,N′-disuccinic acid (EDDS), and any combinations thereof.

The following non-limiting formulation examples illustrate representative pharmaceutical compositions that may be prepared in accordance with the present disclosure.

Formulation 1—Tablets. A compound of the present disclosure may be admixed as a dry powder with a dry binder in an approximate 1:2 weight ratio. Additional diluent may be added as necessary, and a minor amount of magnesium stearate is added as a lubricant. The mixture is formed into 150-1500 mg tablets (50-500 mg of active compound per tablet) in a tablet press.

Formulation 2—Capsules. A peptoid compound described herein may be admixed as a dry powder with a starch diluent in an approximate 1:1 weight ratio. The mixture is filled into empty capsule shells (50-500 mg of peptoid compound per capsule).

Formulation 3—Liquid. A peptoid compound described herein (50-500 mg) may be admixed with sucrose (1.75 g) and xanthan gum (4 mg) and the resultant mixture may be blended, passed through a No. 10 mesh U.S. sieve, and then mixed in water. Sodium benzoate (10 mg), flavor, and color are diluted with water and added with stirring. Sufficient water may then be added to produce a total volume of 5 mL.

Formulation 4—Tablets. A peptoid compound described herein may be admixed as a dry powder with a dry gelatin binder in an approximate 1:2 weight ratio. A minor amount of magnesium stearate is added as a lubricant. The mixture is formed into 450-900 mg tablets (150-300 mg of active compound) in a tablet press.

Formulation 5—Injection. A peptoid compound described herein may be dissolved or suspended in a buffered sterile saline injectable aqueous medium to a concentration of approximately 0.1-5 mg/mL.

Formulation 6—Topical. Stearyl alcohol (250 g) and a white petrolatum (250 g) may be melted at about 75° C. and then a mixture of a peptoid compound described herein (1-100 g) methylparaben (0.25 g), propylparaben (0.15 g), sodium lauryl sulfate (10 g), and propylene glycol (120 g) dissolved in water (about 370 g) may be added and the resulting mixture is stirred until it congeals.

Formulation 7—Intranasal. To prepare 1 L of a 25 mM phosphate buffer, dissolve 0.6 g of potassium phosphate dibasic and 2.93 g of potassium phosphate monobasic in 800 mL of deionized (DI) water. Second, slowly add 2 g of both glycerin and 2 g sorbitol while mixing with an overhead mixer. Stir until the solution is clear and free from undissolved particulates. Next, slowly add 50 g of hypromellose while stirring with an overhead mixer until dissolved for a 5% solution. Then, add 1 g of EDTA as a preservative for the multidose solution, and finally add 100 mg of the peptoid for a 0.01% (% w/v) solution. The solution is diluted to 1 L with DI water, the pH adjusted with a 1N NaOH or 1N HCl solution to a pH of 5.0-7.0 and stirred until a clear solution is obtained.

The peptoid may be included in the formulation over a range of 0.005%-5%. Alternate buffer agents include histidine buffer for pH control in the physiological range, and may be utilized over a molarity range of 10 mM-100 mM. Alternate viscosity increasing agents include, but are not limited to, carbomers, polyvinylpyrrolidone (PVP), hydroxyethylcellulose (HEC), and poloxamers, and may be present in a range of 2-10%. Osmolality increasing agents can also include, but are not limited to, sorbitol, sodium citrate, or dextrose, and may be included at 1-5%. Taste masking agents can include, but are not limited to, sucrose and/or other sugars and may be present at 1-5%. Preservatives may be included in the range of 0.05%-2%, and can also include, but are not limited to, benzalkonium chloride and sodium benzoate.

Formulation 8—Subcutaneous Injection. To prepare 1 L of a 25 mM phosphate buffer, dissolve 0.6 g of potassium phosphate dibasic and 2.93 g of potassium phosphate monobasic in 800 mL of deionized (DI) water. The solution is diluted to 1 L with DI water, the pH adjusted with a 1N NaOH or 1N HCl solution to a target pH of 6.5 (range 6.0-7.0) and stirred until a clear solution is obtained. Slowly add 1 g of peptoid and stir until completely dissolved for a target concentration of 1 mg/mL (0.1% w/v). This solution can be sterilized by using a 0.22 μm filter and stored in a sterile container with closure until use.

Additional example peptoid formulations are described in Example 3.

The peptoid may be included in the formulation over a range of 0.005%-5%. Alternate buffer agents include histidine buffer for pH control in the physiological range, and may be utilized over a molarity range of 10 mM-100 mM. Preservatives may be included in the range of 0.05%-2%, and can also include, but are not limited to, benzalkonium chloride and sodium benzoate.

In some embodiments, the compositions described herein may be formulated as mixtures of one or more peptoids. For example, these mixtures may comprise peptoids in various molar ratios, such as 0.01:0.99 to 0.99:0.01, or any ratio in between. In some embodiments, the effective amount may be from 1-1000 mg/day, with a preferred embodiment of 25-750 mg/day, or a more preferred embodiment of 50-500 mg/day, or an even more preferred embodiment of 100-400 mg/day.

In some embodiments, a composition may comprise a peptoid compound described herein in mixtures or combinations with other agents, such as known antibiotic compounds. In some embodiments, the peptoid compounds of the present disclosure may act synergistically with the known antibiotic compounds, so that the resulting composition demonstrates improved effectiveness.

In some embodiments, the subject may be a vertebrate animal. In some embodiments, the subject may be a mammal. In some embodiments, the subject may be a primate. In some embodiments, the subject may be a human. In some embodiments, the methods disclosed herein have veterinary applications and can be used to treat non-human animals, such as wild, domestic, or farm animals, including, but not limited to, cattle, sheep, goats, pigs, dogs, cats, and poultry.

“Treating” or “treatment” of refers, in some embodiments, to aiding in healing a pathological condition, ameliorating the pathological condition (e.g., arresting or reducing worsening of the pathological condition or at least one of the symptoms related to the pathological condition). In some embodiments “treating” or “treatment” refers to ameliorating at least one physical parameter, which may not be discernible by the subject. In some embodiments, “treating” or “treatment” refers to modulating the pathological condition, either physically, (e.g., stabilization of a discernible symptom), physiologically, (e.g., stabilization of a physical parameter), or both.

“Preventing” or “prevention” as used herein refers to a reduction in risk of acquiring a pathological condition (e.g., causing at least one of the clinical symptoms of the pathological condition not to develop in a subject not yet exposed to or predisposed or susceptible to the pathological condition, and not yet experiencing or displaying symptoms of the pathological condition).

In some embodiments, the term “treating” or “treatment” of a pathological condition encompasses preventing or inhibiting the pathological condition. In some embodiments, an infection may be a viral infection, a bacterial infection, a fungal infection, or any combination thereof.

In some embodiments, a compositions and methods described herein may be combined with other compositions and methods, including known active ingredients, compositions and methods, either in the same composition, or administered separately. In some embodiments, the compositions and methods of the present disclosure may act synergistically with the other active ingredients, compositions and methods, so that the resulting compositions and/or methods demonstrate improved effectiveness.

In some embodiments, the compositions and methods described herein may be useful for applications as consumer care products, such as over-the counter products, or products prescribed by a healthcare professional. In some embodiments, the compositions and methods described herein may be useful for applications in extreme environments such as in the battlefield, for military use, or in emergency scenarios, such as in first aid kits, for home use, for clinical use, for first responder uses, and the like.

In some embodiments, the compositions described herein are non-toxic to human cells, show improved tolerability, improved efficacy, or any combinations thereof, compared to previously existing compositions and products for treating or preventing a pathological condition described herein.

### EXAMPLES

The present examples are provided for illustrative purposes only. They are not intended to and should not be interpreted to encompass the full breadth of the invention.

### Example 1. Preparation of Peptoid Compounds

Peptoid compounds can be prepared using a sub-monomer protocol, on Rink Amide MBHA Resin. Example sub-monomer protocols are described in Zuckermann, R. N., Kerr, J. M., Kent, S. B. H., & Moos, W. H. (1992) J. Am. Chem. Soc., 114, 10646-10647 and in U.S. Pat. Nos. 8,445,632 and 6,887,845, the entireties of which are incorporated herein by reference. The following briefly describes a non-limiting example preparation of peptoid compounds. Typically, the starting reagents are bromoacetic acid and a small set of primary amines that are readily available commercially. The crude peptoid products are then cleaved from the resin and sidechain protective groups were removed in one step by acidolysis. The resulting residue is then resolubilized and lyophilized twice to produce peptoids as a dry powder. The peptoid products are then purified by HPLC to produce peptoids in powder form, with hydrochloride as the counter ion. Peptoid compounds are stored as dry powder at −20° C. and protected from light prior to preparation of stock solutions. Non-limiting examples of peptoid compound are shown in Table 1.

TABLE 1
Example Peptoid compounds

| | | Molecular | | |
|---|---|---|---|---|
| | | weight | | |
| Peptoid | | without | | |
| compound | | HCl salt | Molecular | Counter |
| name | Peptoid Sequence | (g/mol) | Formula | Ion |
| MXB- | H-NLys-NSpe(p- | 1233.78 | C₅₂H₆₇Br₄N₉O₆ | Hydro- |
| 24,656 | Br)-NSpe(p-Br)- | (1343.15 | | chloride |
| | NLys-NSpe(p-Br)₅- | with | | |
| | NSpe(p-Br)-NH₂ | HCl salt) | | |
| MXB- | H-Ntridec-NLys- | 835.19 | C₄₇H₇₈N₈O₅ | Hydro- |
| 22,510 | Nspe-Nspe-NLys- | (944.56 | | chloride |
| | NH₂ | with | | |
| | | HCl salt) | | |
| MXB- | H-Ndec-NLys- | 1273.31 | C₆₄H₉₂Br₂N₁₀O₇ | Hydro- |
| 27,369 | Nspe-Nspe(p-Br)- | (1382.68 | | chloride |
| | NLys-Npse- | with | | |
| | Nspe(p-Br)-NH₂ | HCl salt) | | |
| MXB- | H-Ndodec-NLys- | 821.17 | C₄₆H₇₆N₈O₅ | Hydro- |
| 25,605 | Nspe-Nspe-NLys- | (930.53 | | chloride |
| | NH₂ | with | | |
| | | HCl salt) | | |
| MXB- | H-Ntetradec-NLys- | 849.22 | C₄₈H₈₀N₈O₅ | Hydro- |
| 24,816 | Nspe-Nspe-NLys- | (958.58 | | chloride |
| | NH₂ | with | | |
| | | HCl salt) | | |
| MXB- | H-Ndec-NLys- | 1401.49 | C₇₀H₁₀₄Br₂N₁₂O₈ | Hydro- |
| 25,739 | Nspe-Nspe(p-Br)- | (1547.32 | | chloride |
| | NLys-Nspe-Nspe(p- | with | | |
| | Br)-NLys-NH₂ | HCl salt) | | |

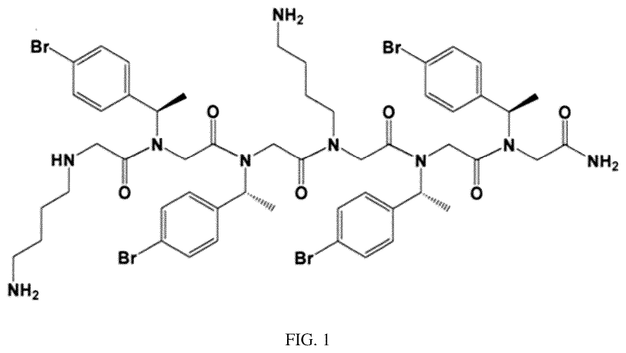

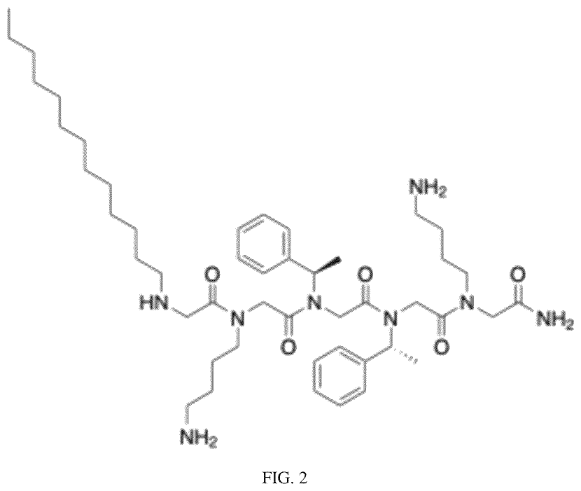

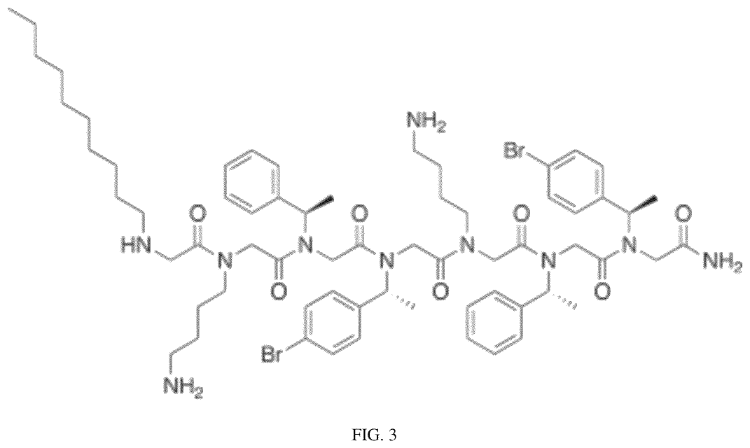

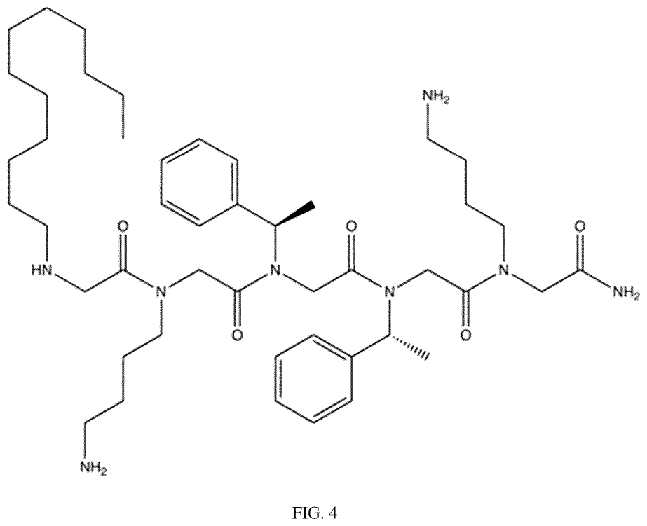

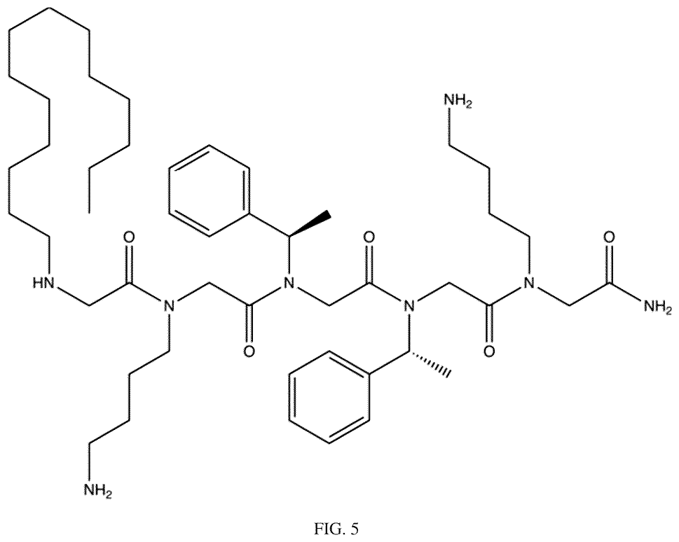

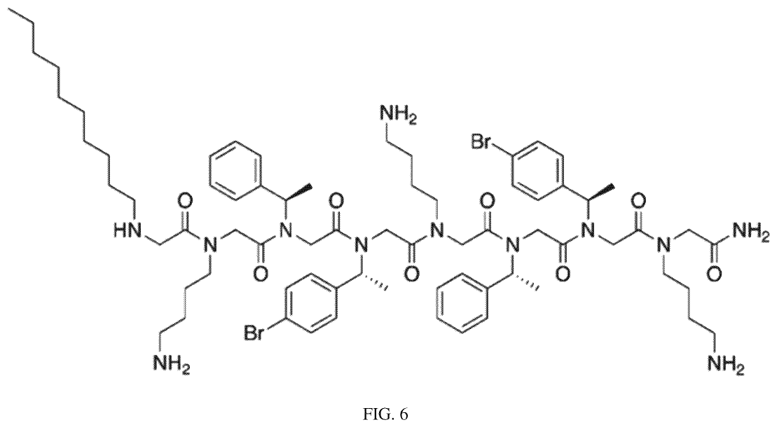

Chemical structures of the peptoid compounds listed in Table 1 are shown in FIG. 1-FIG. 6.

An initial stock concentration of each peptoid compound is prepared in tubes at 2 mg/ml in phosphate-buffered saline (PBS) pH 7.4 (Gibco; cat no. 10010023). Initial dissolution of lyophilized peptoid compound powders to create a stock solution is performed by gentle mixing by inverting the stock solution tube several times), followed by checking for turbidity, precipitation, or aggregate before proceeding to the next steps. If gentle inversion is insufficient to achieve a solution, the stock solution tube is briefly vortexed. The stock solution is then checked for any undissolved particulate, aggregates, or precipitation before proceeding to the next step. If gentle inversion and vortexing is insufficient to achieve a solution, the stock solution tube is briefly sonicated for 15-60 seconds. The stock solution is then checked again for any undissolved particulate, aggregates, or precipitation before proceeding to the next step. If turbidity, precipitation, or aggregate is observed at the initial stock concentration, the initial stock concentration is solubilized by diluting further in PBS to 1 mg/ml. Aliquots of the stock solutions are dispensed in polypropylene vials, protected from light, and stored at −20° C. or −80° C. prior to use.

Before testing, the aliquots are carefully observed for any signs of turbidity, precipitation or aggregate during sample preparation and are mixed, vortexed, or sonicated as needed.

### Example 2. Testing In Vitro LPS-Binding Activity of Peptoid Compounds

This Example describes example procedures for testing in vitro LPS-binding activity of peptoid compounds.

Objective. To visualize the binding interaction between peptoid compounds and LPS.

### Materials

### Peptoid Compounds

Lipopolysaccharide (LPS) from a representative Gram-negative bacterium.
Fluorescently labeled peptoid compounds or a fluorescent probe that can bind to the peptoid compounds.
Fluorescent dye for LPS, ensuring it has a distinct emission spectrum from the peptoid compound's fluorescent label or probe.
Quartz cuvettes for fluorescence spectroscopy.
Fluorescence microscope equipped with the appropriate filters.
Fluorescence spectroscopy equipment.
Buffer solution mimicking physiological conditions.

### Experimental Steps

Step 1: Preparation. Prepare solutions of fluorescently labeled peptoid compounds and LPS in buffer. Ensure concentrations are within the dynamic range for fluorescence detection and are relevant to physiological conditions.

Step 2: Fluorescence Labeling (if not pre-labeled). If peptoid compound and LPS are not pre-labeled, conjugate them with fluorescent probes ensuring that the probes do not interfere with the binding interaction. Choose probes with distinct excitation/emission spectra to differentiate between peptoid compound and LPS signals.

Step 3: Binding Assay. Mix peptoid compound and LPS in a quartz cuvette and incubate at 37° C. for an appropriate time to allow binding. The volume and concentrations should be determined based on preliminary experiments to optimize signal detection.

Control samples: peptoid compound or LPS alone with their respective fluorescent labels.

Step 4: Fluorescence Spectroscopy. Measure fluorescence intensity of the mixtures at both peptoid compound and LPS wavelengths to detect any changes indicative of binding. An increase in fluorescence resonance energy transfer (FRET) efficiency between the peptoid compound and LPS labels suggests interaction.

Step 5: Fluorescence Microscopy. Prepare slides with peptoid-LPS mixtures for fluorescence microscopy. Use filters appropriate for the fluorescent labels used. Image the samples to visualize colocalization of peptoid and LPS, indicating binding. Use control samples to establish baseline fluorescence and specificity of interaction.

### Data Analysis

Compare fluorescence intensity and microscopy images between control and test samples. Binding is indicated by changes in FRET efficiency (spectroscopy) and colocalization of fluorescence signals (microscopy).

Analyze the specificity and efficiency of binding through varying concentrations of peptoid compound and LPS.

### CONCLUSION

This example experiment provides both qualitative and quantitative data on the interaction between a peptoid compound and LPS. It is expected that at least one peptoid compound described herein will show LPS-binding activity. Peptoid compounds capable of binding LPS are expected to prevent or decrease one or more negative effects of LPS in pathological conditions caused by or associated with LPS in humans and/or animals.

### Considerations for Assays

Ensure fluorescent labeling does not affect the biological activity of peptoid compound or LPS.
Verify the specificity of the interaction through control experiments, including competition assays with unlabeled peptoid compound or LPS.
Adjust concentrations based on preliminary experiments to optimize signal-to-noise ratios.

Additional experiments for testing in vitro LPS-binding activity of peptoid compounds can include, without limitation, experiments to determine a dissociation constant of a peptoid compound and an LPS. Persons of ordinary skill in the art will be able to perform such experiments to determine dissociation constants upon reading the present disclosure, in view of the knowledge in the art.

The dissociation constant (Kd) represents the equilibrium constant for the dissociation of a complex into its constituent parts and may be used to quantify the strength of binding between a ligand (such as lipopolysaccharide, LPS) and an LPS-binding protein or a peptoid compound.

The dissociation constant can vary depending on the specific LPS-binding protein or peptoid, and experimental conditions. However, in general, the dissociation constant for the binding of LPS to LPS-binding proteins typically falls within the range of nanomolar to micromolar concentrations.

For example, studies have reported dissociation constants for the binding of LPS to various LPS-binding proteins, such as LPS-binding protein (LBP), CD14, and soluble Toll-like receptors (TLRs), in the range of approximately 10⁻⁸ to 10⁻⁶ M.

In some embodiments, the dissociation constant for the binding of a peptoid compound to an LPS can be determined empirically, and can be, e.g., from 10⁻⁹ to 10⁻³ M.

### Example 3. Example Formulations of Nentoid Compounds

The peptoids described herein may be formulated into a wide variety of dosage forms for topical, targeted local delivery, or systemic delivery.

### Topical Formulation Approaches

Topical formulation approaches include powder, solution, suspension, semisolids, or infused into a bandage or other dressing material. Powder formulations include, but are not limited to, powder, granulation, pellets, or mini tablets. These powder dosage forms may be packaged or contained in a simple stick pack, sachet, vial, spray, shaker bottle, or multi-use bottle. The solution formulations may be provided as a solution, granules or powder for reconstitution, disintegrating tablet for dissolution and reconstitution, or incorporated into a spray bottle, with or without materials to provide a scaffold or topical bandage. The suspension formulations include, but are not limited to, aqueous suspension, suspension in another solvent, granules or powder for suspension, or disintegrating tablet for resuspension. These suspension dosage forms may be packaged or contained in a simple stick pack, sachet, vial, shaker bottle, or multi-use bottle. Semisolid formulation approaches include, but are not limited to, creme, gel, ointment, lotion, paste, balm, salve, emulsion, suppository (e.g. embedded in wax or polymer that liquifies at body temperature), spray, including spray on bandages, foam, including spray on foams, or film. Peptoids can also be infused into dressings including gauze, bandages, among others. Peptoids may be administered using a disintegrating tablet, drug eluting tablet or tablets, drug eluting beads or granules, or implantable, self dissolving sheet, wafer, block or suppository (e.g. embedded in wax or polymer that liquifies at body temperature), or thin wafer inserted for drug elution at a local site.

### Other Delivery Methods

In addition to topical formulation approaches, alternate administration approaches of the peptoids may be employed. These approaches include injection for local delivery, injection for systemic delivery, transdermal patch or other transdermal approach, intravenous, intranasal, intraocular, aural delivery, sublingual, buccal, or oral delivery, including immediate-release dosage forms, as well as modified release. Injection delivery methods may include intraperitoneal, subcutaneous, intramuscular, intrathecal, or intravenous.

### Example Powder Formulation

The peptoids may be administered as a simple powder as a standalone drug or with additional excipients to improve flowability or other processing requirements. This powder may be filled into a hard gelatin capsule and subsequently filled into bottles, or packaged in a sachet, stick pack, vial, or other container to aid in portability and ease of administration. This powder formulation may be applied directly, or dissolved in an aqueous vehicle for topical administration. A powder for topical administration may be prepared using the following formula:

### Formulation 1

Drug Substance—600 g (active ingredient)
Microcrystalline cellulose—100 g (processing aid—flowability)
Lactose—300 g (processing aid—flowability)

Step 1—blend the microcrystalline cellulose and lactose in a suitable blender and blend for 10 minutes

Step 2—add the drug substance and blend for an additional 10 minutes, or until the drug is uniformly distributed throughout the blender.

Step 3—discharge the powder blend from the blender into a suitable bin or container to store until the filling operation.

Step 4—fill the appropriate amount of blend into each package for storage, transfer, and administration.

### Example 2—Granulation

The drug substance powder can also be incorporated into a granulation that can produce a particle with improved flowability and density relative to the powder in Example 1. This granulation can be prepared either dry, or in the presence of water or other solvent. The binder may be added either wet (in the granulation solution) or dry with the rest of the materials. If water or other solvent is used, the blend is dried in a suitable pharmaceutical drier, such as a vacuum oven, forced air oven, or fluid bed drier. This granulation may be filled into a hard gelatin capsule and subsequently filled into bottles, or packaged in a sachet, stick pack, vial, or other container to aid in portability and ease of administration. This granulation formulation may be applied directly, or dissolved in an aqueous vehicle for topical administration. A granule formulation that may be used for topical application or dissolution into a topical solution may be prepared using the following formula:

### Formulation 2

Drug substance—700 g (active ingredient)
Microcrystalline cellulose—200 g (diluent and processing aid)
Povidone—100 g (binder)
Water (processing aid; removed during processing)

Step 1—add the active and excipients to a suitable pharmaceutical mixer or granulator such as a planetary mixer, high-shear granulator, fluid bed granulator, or extruder.

Step 2—slowly add the water while the mixer is operating until all the water has been added.

Step 3—continue the granulation step until the granulation endpoint is achieved.

Step 4—discharge the wet mass into a container suitable to hold the material until drying.

Step 5—charge the wet mass into a suitable drier and dry until the endpoint of less than 2% water is reached.

Step 6—discharge the dried granulation into a suitable bin or container to store until the filling operation.

Step 7—fill the appropriate amount of granulation into each package for storage, transfer, and administration.

Additional formulation examples are shown below, and follow a similar procedure for preparation.

### Formulation 3

Drug substance—700 g (active ingredient)
Microcrystalline cellulose—275 g (diluent and processing aid)
Povidone—75 g (binder)
Croscarmellose sodium—50 g (disintegrant)
Water (processing aid; removed during processing)

### Formulation 4

Drug substance—600 g (active ingredient)
Microcrystalline cellulose—300 g (diluent and processing aid)
Hydroxypropyl cellulose—100 g (binder)
Water (processing aid; removed during processing)

### Formulation 5

Drug substance—800 g (active ingredient)
Microcrystalline cellulose—125 g (diluent and processing aid)
Hydroxypropyl cellulose—50 g (binder)
Croscarmellose sodium—25 g (disintegrant)
Water (processing aid; removed during processing)

### Example 3—Pellets

The drug substance powder can also be incorporated into a pellet that can produce a particle with improved flowability and density relative to the powder in Example 1, and better flowability than the granulations in Example 2. These pellets may be filled into a hard gelatin capsule and subsequently filled into bottles, or packaged in a sachet, stick pack, vial, or other container to aid in portability and ease of administration. Pellet formulations that may be used for topical application or dissolution into a topical solution may be prepared using similar formulations to those shown in Example 2, with the addition of 2 processing steps. This pellet formulation may be applied directly, or dissolved in an aqueous vehicle for topical administration. Pellet formulation examples are shown here:

### Formulation 6

Drug substance—700 g (active ingredient)
Microcrystalline cellulose—200 g (diluent and processing aid)
Povidone—100 g (binder)
Water (processing aid; removed during processing)

### Formulation 7

Drug substance—700 g (active ingredient)
Microcrystalline cellulose—275 g (diluent and processing aid)
Povidone—75 g (binder)
Croscarmellose sodium—50 g (disintegrant)
Water (processing aid; removed during processing)

### Formulation 8

Drug substance—600 g (active ingredient)
Microcrystalline cellulose—300 g (diluent and processing aid)
Hydroxypropyl cellulose—100 g (binder)
Water (processing aid; removed during processing)

### Formulation 9

Drug substance—800 g (active ingredient)
Microcrystalline cellulose—125 g (diluent and processing aid)
Hydroxypropyl cellulose—50 g (binder)
Croscarmellose sodium—25 g (disintegrant)
Water (processing aid; removed during processing)

Step 1—add the active and excipients to a suitable pharmaceutical mixer or granulator such as a planetary mixer, high-shear granulator, fluid bed granulator, or extruder.

Step 2—slowly add the water while the mixer is operating until all the water has been added.

Step 3—continue the granulation step until the granulation endpoint is achieved.

Step 4—Load the wet mass into a suitable extruder and extrude using a screen with apertures between 300 μm and 800 μm.

Step 5—extrude the wet mass and introduce the extrudate into the marumerizer for pellet formation and spheronization.

Step 6—discharge the wet mass of pellets into a container suitable to hold the material until drying.

Step 7—charge the wet mass into a suitable drier and dry until the endpoint of less than 2% water is reached.

Step 8—discharge the dried pellets into a suitable bin or container to store until the filling operation.

Step 9—fill the appropriate amount of pellets into each package for storage, transfer, and administration.

### Example 4—Mini Tablets

The drug substance powder can also be incorporated into minitablets that can produce a particle with similar performance characteristics as a pellet. Minitablets offer another dry formulation approach, where a solvent may not be required. These minitablets typically have a diameter on the order of 500-2000 μm may be filled into a hard gelatin capsule and subsequently filled into bottles, or packaged in a sachet, stick pack, vial, or other container to aid in portability and ease of administration. This minitablet formulations may be applied directly, or dissolved in an aqueous vehicle for topical administration. Minitablet formulation examples are shown here:

### Formulation 10

Drug substance—600 g (active ingredient)
Microcrystalline cellulose—300 g (diluent)
Lactose—100 g (diluent)
Hypromellose (HPMC)—50 g (binder)
Colloidal silicon dioxide—50 g (glidant)
Magnesium stearate—5 g (lubricant)

### Formulation 11

Drug substance—600 g (active ingredient)
Silicified microcrystalline cellulose—400 g (diluent)
Hypromellose (HPMC)—50 g (binder)
Colloidal silicon dioxide—50 g (glidant)
Magnesium stearate—5 g (lubricant)

### Formulation 12

Drug substance—600 g (active ingredient)
Silicified microcrystalline cellulose—400 g (diluent)
Polyvinylpyrrolidone (PVP)—50 g (binder)
Colloidal silicon dioxide—50 g (glidant)
Magnesium stearate—5 g (lubricant)

### Formulation 13

Drug substance—700 g (active ingredient)
Silicified microcrystalline cellulose—250 g (diluent)
Polyethylene glycol (PEG)—50 g (binder)
Colloidal silicon dioxide—50 g (glidant)
Magnesium stearate—5 g (lubricant)

### Example 7—Cream

A topical cream formulation may be prepared using the following formula for the preparation of a batch of approximately 1 Kg:

### Formulation 14

Methyl paraben—0.25 g (preservative)
Propyl paraben—0.15 g (preservative)
Polysorbate 60-10 g (emulsifier)
Propylene glycol—120 g (viscosity modifier)
Stearyl alcohol—200 g (oleaginous phase)
White petrolatum—200 g (oleaginous phase)
Purified water—470 g (aqueous base)

Step 1—mix the stearyl alcohol and white petrolatum and heat to approximately 75° C. to melt the base.

Step 2—Dissolve the remaining excipients in the purified water by stirring until a solution is obtained.

Step 3—Add approximately 5 g of drug substance to the purified water solution and mix for 5 additional minutes to dissolve the drug to manufacture a 5% ointment.

Step 4—Slowly incorporate the aqueous solution to the oleaginous base and mix until it is well mixed.

Step 5—Fill the cream into a suitable package such as a tube or pump bottle.

### Example 8—Gel

Topical gels can be used for sustained-release of actives, provide lubrication, and a carrier of pharmaceutical agents. Hydrogels are water-based and are less oily than creams or ointments, as well as exhibit excellent spreading properties, and may exhibit a higher retention time on the skin. Hydrogels can be simple formulations and may provide for a higher drug capacity than oil based formulations due to the high aqueous solubility of the drug substance. Gel formulation examples are shown here:

### Formulation 15

Drug Substance—50 g (active)
Carbopol—300 g (polymer/viscosity)
Purified Water—650 g (solvent)

### Formulation 16

Drug Substance—50 g (active)
Sodium carboxymethylcellulose—400 g (polymer/viscosity)
Purified Water—550 g (solvent)

### Formulation 17

Drug Substance—50 g (active)
Hypromellose—200 g (polymer/viscosity)
Purified Water—750 g (solvent)

Step 1—Slowly add the polymer to the purified water while stirring slowly using a suitable mixer such as a Silverson mixer. Continue to mix until the polymer exhibits a lump-free dispersion.

Step 2—Slowly add the drug substance to the polymer dispersion and mix until dissolved.

Step 3—Fill the gel into a suitable package such as a tube or pump bottle.

### Example 9—Ointment

Hydrophilic ointment may be prepared using the following formula for the preparation of about 1 Kg of base:

### Formulation 18

Methyl paraben—0.25 g (preservative)
Propyl paraben—0.15 g (preservative)
Sodium lauryl sulfate—10 g (emulsifier)
Propylene glycol—120 g (viscosity modifier)
Stearyl alcohol—250 g (oleaginous phase)
White petrolatum—250 g (oleaginous phase)
Purified water—370 g (aqueous base)

Step 1—mix the stearyl alcohol and white petrolatum and heat to approximately 75° C. to melt the base.

Step 2—Dissolve the remaining excipients in the purified water by stirring until a solution is obtained.

Step 3—Add approximately 1 g of drug substance to the purified water solution and mix for 5 additional minutes to dissolve the drug to manufacture a 1% ointment.

Step 4—Slowly incorporate the aqueous solution to the oleaginous base and mix until it congeals.

Step 5—Fill the ointment into a suitable package.

### Example 10—Sterile Solution for Subcutaneous or Intramuscular Administration

The drug may be incorporated into a solution for delivery via intramuscular (IM), subcutaneous (SC), or intravenous (IV) administration. Formulations designed to deliver active drug substances via the IM or SC route will generally have similar concentrations and volumes of administration. Formulations intended to provide 1 L of drug formulation for SC or IM administration are shown here:

Formulation 19-10% (100 mg/mL)
Drug Substance—100 g (active)
Phosphate Buffer solution—1 L (solvent)
pH adjustment—0.1 N NaOH or 0.1 N HCl (pH adjustment)
   Formulation 20-5% (50 mg/mL)
Drug Substance—50 g (active)
Phosphate Buffer solution—1 L (solvent)
pH adjustment—0.1 N NaOH or 0.1 N HCl (pH adjustment)
   Formulation 21-1% (10 mg/mL)
Drug Substance—10 g (active)
Phosphate Buffer solution—1 L (solvent)
pH adjustment—0.1 N NaOH or 0.1 N HCl (pH adjustment)
   Formulation 22-0.1% (1 mg/mL)
Drug Substance—1 g (active)
Phosphate Buffer solution—1 L (solvent)
pH adjustment—0.1 N NaOH or 0.1 N HCl (pH adjustment)
   Formulation 23-0.05% (500 μg/mL)
Drug Substance—0.5 g (active)
Phosphate Buffer solution—1 L (solvent)
pH adjustment—0.1 N NaOH or 0.1 N HCl (pH adjustment)

Step 1—Slowly add the drug substance to the buffer solution while stirring.

Step 2—Continue stirring until a clear solution is obtained.

Step 3—Measure the pH of the solution, and adjust to a pH of 6.5-7.5 using the dilute HCl or NaOH solution.

Step 4—Sterile filtration using a 0.22 μm filter, and fill into a sterile syringe for a pre-filled syringe drug-device combination.

The above disclosure contains various examples of peptoid compound compositions and methods of use thereof. Aspects of these various examples may all be combined with one another, even if not expressly combined in the present disclosure, unless they are clearly mutually exclusive.

In addition, various example materials are discussed herein and are identified as examples, as suitable materials, and as materials included within a more generally described type of material, for example by use of the term “including” or “such-as.” All such terms are used without limitation, such that other materials falling within the same general type exemplified but not expressly identified may be used in the present disclosure as well.

Furthermore, unless it is otherwise clear that a single entity is intended, terms such as “a,” “an,” and “the” are not intended to refer to only a singular entity and include the general class of which a specific example is described for illustration. In addition, unless it is clear that a precise value is intended, numbers recited herein should be interpreted to include variations above and below that number that may achieve substantially the same results as that number, or variations that are “about” the same number. Finally, a derivative as disclosed herein may include a chemically modified molecule that has an addition, removal, or substitution of a chemical moiety of the parent molecule.

It is understood the use of the alternative (e.g., “or”) herein is taken to mean either one or both or any combination thereof of the alternatives. The term “and/or” used herein is to be taken mean specific disclosure of each of the specified features or components with or without the other. For example, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

As used herein, terms “comprising”, “including”, “having” and “containing”, and their grammatical variants, as used herein are intended to be non-limiting so that one item or multiple items in a list do not exclude other items that can be substituted or added to the listed items. It is understood that wherever aspects are described herein with the language “comprising,” otherwise analogous aspects described in terms of “consisting of” and/or “consisting essentially of” are also provided.

Various compositions may be identified by trade name in this application. All such trade names refer to the relevant composition or instrument as it existed as of the earliest filing date of this application, or the last date a product was sold commercially under such trade name, whichever is later. One of ordinary skill in the art will appreciate that variant compositions and instruments sold under the trade name at different times will typically also be suitable for the same uses.

The above disclosed subject matter is to be considered illustrative, and not restrictive, and the appended claims are intended to cover all such modifications, enhancements, and other implementations which fall within the true spirit and scope of the present disclosure. Thus, to the maximum extent allowed by law, the scope of the present disclosure is to be determined by the broadest permissible interpretation of the following claims and their equivalents and shall not be restricted or limited by the foregoing detailed description.

1. A method of treating or preventing a pathological condition in a subject, wherein the pathological condition is caused by or associated with the presence of a lipopolysaccharide (LPS) in the subject, comprising:
administering to the subject an effective amount of one or more peptoid compounds adapted to bind to the LPS.

2. The method of claim 1, wherein the LPS is produced by a Gram-negative bacteria selected from the group consisting of Escherichia (e.g., Escherichia coli), Salmonella (e.g., Salmonella enterica), Klebsiella (e.g., Klebsiella pneumoniae), Pseudomonas (e.g., Pseudomonas aeruginosa), Vibrio (e.g., Vibrio cholerae), Helicobacter (e.g., Helicobacter pylori), Neisseria (e.g., Neisseria meningitidis or Neisseria gonorrhoeae), Bordetella (e.g., Bordetella pertussis, Yersinia (e.g., Yersinia pestis, Yersinia enterocolitica, or Yersinia pseudotuberculosis), Haemophilus (e.g., Haemophilus influenzae), Legionella (e.g., Legionella pneumophila), Campylobacter (e.g., Campylobacter jejuni or Campylobacter coli), Acinetobacter (e.g., Acinetobacter baumannii), Brucella (e.g., Brucella abortus, Brucella melitensis, or Brucella suis), Francisella (e.g., Francisella tularensis), Bacteroides, Shigella (e.g., Shigella flexneri, or Shigella sonnei), Burkholderia (e.g., Burkholderia cepacia or Burkholderia pseudomallei), Pasteurella (e.g., Pasteurella multocida), Proteus (e.g., Proteus mirabilis or Proteus vulgaris), Serratia (e.g., Serratia marcescens), Treponema (e.g., Treponema pallidum), Enterobacter (e.g., Enterobacter cloacae or Enterobacter aerogenes), Moraxella (e.g., Moraxella catarrhalis), Fusobacterium (e.g., Fusobacterium nucleatum), Actinobacillus (e.g., Actinobacillus actinomycetemcomitans), Tannerella (e.g., Tannerella forsythia), Prevotella, Fusobacterium (e.g., Fusobacterium nucleatum), Desulfovibrio, and Capnocytophaga.

3. The method of claim 1, wherein the pathological condition is selected from the group consisting of Rheumatoid Arthritis, Systemic Lupus Erythematosus, Inflammatory Bowel Disease, Type 1 Diabetes, Multiple Sclerosis, Psoriasis, Ankylosing Spondylitis, Sepsis, Septic shock, Acute respiratory distress syndrome, Multiple organ dysfunction syndrome, Endotoxemia, Neuroinflammation (e.g., in Alzheimer's disease, Parkinson's disease, multiple sclerosis, or depression), Periodontal disease, Chronic inflammatory diseases (rheumatoid arthritis, lupus erythematosus, or IBD), Cardiovascular diseases, Liver diseases (e.g., alcoholic liver disease, non-alcoholic fatty liver disease (NAFLD), or liver cirrhosis), Metabolic disorders (e.g., obesity, insulin resistance, and type 2 diabetes), Acute lung injury, Acute kidney injury, Neonatal disorders (e.g., neonatal sepsis or necrotizing enterocolitis), Autoimmune diseases (e.g, systemic lupus erythematosus (SLE), multiple sclerosis (MS), or autoimmune thyroiditis), Gastrointestinal disorders (e.g., irritable bowel syndrome (IBS), inflammatory bowel disease (IBD), or celiac disease), Reproductive disorders (e.g., preterm labor, fetal growth restriction, or infertility), Psychiatric disorders (e.g., depression, anxiety, or schizophrenia), Chronic obstructive pulmonary disease, Chronic kidney disease, end-stage renal disease, Cholestasis, Pulmonary hypertension, Acute pancreatitis, Osteoarthritis, Cardiomyopathy, Alcoholic liver disease, Gastroesophageal reflux disease, Gastrointestinal cancers, Autoimmune hepatitis, Preterm birth, neonatal complications, Asthma, Necrotizing enterocolitis, Non-alcoholic fatty liver disease, non-alcoholic steatohepatitis, liver cirrhosis, hepatocellular carcinoma, Chronic fatigue syndrome, Vascular dementia, Gut-brain axis disorders, Autism spectrum disorder, Obstructive sleep apnea, Interstitial cystitis/bladder pain syndrome, Chronic rhinosinusitis, Amyotrophic lateral sclerosis, Interstitial Lung Disease, Celiac disease, Autoimmune Encephalitis, Gastric Ulcers, Idiopathic Pulmonary Fibrosis, Preeclampsia, Cardiovascular Diseases, Polycystic Ovary Syndrome, Endometriosis, Periodontal Disease, and inflammation associated with bacterial infections.

4. The method of claim 1, wherein the peptoid compound prevents or decreases activity of LPS in the subject.

5. The method of claim 1, wherein the peptoid compound prevents, increases, or decreases binding of the LPS to one or more LPS-binding proteins in the subject.

6. The method of claim 5, wherein the LPS-binding protein is selected from the group consisting of LPS-binding protein, CD14, MD-2, Toll-like receptor 4, Soluble CD14, Lipopolysaccharide and beta-1,3-glucan binding protein, Pentraxins, Surfactant proteins, Lipopolysaccharide-binding protein 2, Bactericidal/permeability-increasing protein, Limulus anti-LPS factor, Plasma lipopolysaccharide-binding protein, Ficolins, Mannose-binding lectin, Lymphocyte antigen 96, Cationic Antimicrobial Peptides, Periplasmic Binding Proteins, Scavenger Receptors, Lipid-Binding Proteins, and Tumor Necrosis Factor Receptor Superfamily Member 6.

7. The method of claim 4, wherein the activity of LPS is decreased by up to 10%, 20%, 30%, 40%, 50%, 60%, 70%, 80%, 90%, 95%, 99%, or more.

8. The method of claim 1, wherein the one or more peptoid compounds are formulated in a composition comprising the one or more peptoid compounds and one or more pharmaceutically acceptable excipients.

9. The method of claim 8, wherein the composition is formulated for topical administration, transdermal administration, transmucosal administration, intraperitoneal administration, subcutaneous administration, intramuscular administration, or intravenous administration to the subject.

10. The method of claim 1, wherein the effective amount is from 1-1000 mg/day, 25-750 mg/day, 50-500 mg/day, or 100-400 mg/day.

11. The method of claim 1, wherein the administration is one, two, three, or four times per day, once per week, once every two weeks, or once per month.

12. A composition for use in a method of treating or preventing a pathological condition in a subject, wherein the pathological condition is caused by or associated with the presence of a lipopolysaccharide (LPS) in the subject, the composition comprising:
an effective amount of one or more peptoid compounds adapted to bind to the LPS.

13. The composition of claim 12, wherein the LPS is produced by a Gram-negative bacteria selected from the group consisting of Escherichia (e.g., Escherichia coli), Salmonella (e.g., Salmonella enterica), Klebsiella (e.g., Klebsiella pneumoniae), Pseudomonas (e.g., Pseudomonas aeruginosa), Vibrio (e.g., Vibrio cholerae), Helicobacter (e.g., Helicobacter pylori), Neisseria (e.g., Neisseria meningitidis or Neisseria gonorrhoeae), Bordetella (e.g., Bordetella pertussis, Yersinia (e.g., Yersinia pestis, Yersinia enterocolitica, or Yersinia pseudotuberculosis), Haemophilus (e.g., Haemophilus influenzae), Legionella (e.g., Legionella pneumophila), Campylobacter (e.g., Campylobacter jejuni or Campylobacter coli), Acinetobacter (e.g., Acinetobacter baumannii), Brucella (e.g., Brucella abortus, Brucella melitensis, or Brucella suis), Francisella (e.g., Francisella tularensis), Bacteroides, Shigella (e.g., Shigella flexneri, or Shigella sonnei), Burkholderia (e.g., Burkholderia cepacia or Burkholderia pseudomallei), Pasteurella (e.g., Pasteurella multocida), Proteus (e.g., Proteus mirabilis or Proteus vulgaris), Serratia (e.g., Serratia marcescens), Treponema (e.g., Treponema pallidum), Enterobacter (e.g., Enterobacter cloacae or Enterobacter aerogenes), Moraxella (e.g., Moraxella catarrhalis), Fusobacterium (e.g., Fusobacterium nucleatum), Actinobacillus (e.g., Actinobacillus actinomycetemcomitans), Tannerella (e.g., Tannerella forsythia), Prevotella, Fusobacterium (e.g., Fusobacterium nucleatum), Desulfovibrio, and Capnocytophaga.

14. The composition of claim 12, wherein the pathological condition is selected from the group consisting of Rheumatoid Arthritis, Systemic Lupus Erythematosus, Inflammatory Bowel Disease, Type 1 Diabetes, Multiple Sclerosis, Psoriasis, Ankylosing Spondylitis, Sepsis, Septic shock, Acute respiratory distress syndrome, Multiple organ dysfunction syndrome, Endotoxemia, Neuroinflammation (e.g., in Alzheimer's disease, Parkinson's disease, multiple sclerosis, or depression), Periodontal disease, Chronic inflammatory diseases (rheumatoid arthritis, lupus erythematosus, or IBD), Cardiovascular diseases, Liver diseases (e.g., alcoholic liver disease, non-alcoholic fatty liver disease (NAFLD), or liver cirrhosis), Metabolic disorders (e.g., obesity, insulin resistance, and type 2 diabetes), Acute lung injury, Acute kidney injury, Neonatal disorders (e.g., neonatal sepsis or necrotizing enterocolitis), Autoimmune diseases (e.g, systemic lupus erythematosus (SLE), multiple sclerosis (MS), or autoimmune thyroiditis), Gastrointestinal disorders (e.g., irritable bowel syndrome (IBS), inflammatory bowel disease (IBD), or celiac disease), Reproductive disorders (e.g., preterm labor, fetal growth restriction, or infertility), Psychiatric disorders (e.g., depression, anxiety, or schizophrenia), Chronic obstructive pulmonary disease, Chronic kidney disease, end-stage renal disease, Cholestasis, Pulmonary hypertension, Acute pancreatitis, Osteoarthritis, Cardiomyopathy, Alcoholic liver disease, Gastroesophageal reflux disease, Gastrointestinal cancers, Autoimmune hepatitis, Preterm birth, neonatal complications, Asthma, Necrotizing enterocolitis, Non-alcoholic fatty liver disease, non-alcoholic steatohepatitis, liver cirrhosis, hepatocellular carcinoma, Chronic fatigue syndrome, Vascular dementia, Gut-brain axis disorders, Autism spectrum disorder, Obstructive sleep apnea, Interstitial cystitis/bladder pain syndrome, Chronic rhinosinusitis, Amyotrophic lateral sclerosis, Interstitial Lung Disease, Celiac disease, Autoimmune Encephalitis, Gastric Ulcers, Idiopathic Pulmonary Fibrosis, Preeclampsia, Cardiovascular Diseases, Polycystic Ovary Syndrome, Endometriosis, Periodontal Disease, and inflammation associated with bacterial infections.

15. The composition of claim 12, wherein the peptoid compound prevents or decreases activity of LPS in the subject.

16. The composition of claim 12, wherein the peptoid compound prevents, increases, or decreases binding of the LPS to one or more LPS-binding proteins in the subject.

17. The composition of claim 16, wherein the LPS-binding protein is selected from the group consisting of LPS-binding protein, CD14, MD-2, Toll-like receptor 4, Soluble CD14, Lipopolysaccharide and beta-1,3-glucan binding protein, Pentraxins, Surfactant proteins, Lipopolysaccharide-binding protein 2, Bactericidal/permeability-increasing protein, Limulus anti-LPS factor, Plasma lipopolysaccharide-binding protein, Ficolins, Mannose-binding lectin, Lymphocyte antigen 96, Cationic Antimicrobial Peptides, Periplasmic Binding Proteins, Scavenger Receptors, Lipid-Binding Proteins, and Tumor Necrosis Factor Receptor Superfamily Member 6.

18. The composition of claim 15, wherein the activity of LPS is decreased by up to 10%, 20%, 30%, 40%, 50%, 60%, 70%, 80%, 90%, 95%, 99%, or more.

19. The composition of claim 12, comprising the one or more peptoid compounds and one or more pharmaceutically acceptable excipients.

20. The composition of claim 12, formulated for topical administration, transdermal administration, transmucosal administration, intraperitoneal administration, subcutaneous administration, intramuscular administration, or intravenous administration to the subject.

21.-22. (canceled)
